(19) United States
(12) Patent Application Publication
(10) Pub. No.: US 2005/0147608 A1
(43) Pub. Date: Jul. 7, 2005

(54) Novel regulatory mechanisms of NF-kappaB

(75) Inventors: Akihide Ryo, Yokohama (JP); Kun Ping Lu, Newton, MA (US)

Correspondence Address: LAHIVE & COCKFIELD, LLP., 28 STATE STREET, BOSTON, MA, 02109, US

(73) Assignee: Beth Israel Deaconess Medical Center, Inc., Boston, MA (US)

(21) Appl. No.: 10/840,827

(22) Filed: May 7, 2004

Related U.S. Application Data
(60) Provisional application No. 60/490,109, filed on Jul. 25, 2003.
(60) Provisional application No. 60/469,542, filed on May 8, 2003.

(51) Int. Cl. A61K 39/395; A61K 48/00; C12N 9/64

(52) U.S. Cl. 424/145.1; 514/44; 435/226; 435/69.1; 435/325; 435/320.1

(57) ABSTRACT

The instant invention pertains to the discovery of two novel regulatory mechanisms of NF-kB. The instant invention demonstrates that NF-kB is regulated by Pin1-catalyzed prolyl isomerization and ubiquitin-mediated proteolysis of p65. Accordingly, the instant invention provides methods for regulating NF-kB, and diseases and disorders associated with NF-kB. Further, the invention provides compositions capable of modulating the activity or expression of NF-kB, Pin1, and/or the proteolysis of p65.

### RELATED APPLICATIONS

This application claims priority to U.S. Provisional Patent Application Ser. No. 60/490,109, filed on Jul. 25, 2003 and U.S. Provisional Patent Application Ser. No. 60/469,542, filed on May 8, 2003, the entire contents of which are incorporated herein by reference.

### GOVERNMENT FUNDING

This invention was made at least in part with support under grant numbers R01GM56230 and GM58556, awarded by the United States National Institute of Health.

### BACKGROUND

The transcription factor NF-kB is activated by degradation of its inhibitor IkB, resulting in its nuclear translocation. However, how nuclear NF-kB is subsequently regulated is not clear and whether its stability is regulated has not been described.

The nuclear factor-kappaB (NF-kB)/Rel family of proteins are inducible transcription factors that play a central role in regulating the expression of a wide variety of genes associated with cell proliferation, immune response, inflammation, cell survival and oncogenesis (Baeuerle and Henkel, 1994; Ghosh and Karin, 2002; Karin et al., 2002; Li and Verma, 2002; Sen and Baltimore, 1986). NF-kB is predominantly a hetrodimeric complex of p65/RelA and p50, although other types of heterodimers have been reported (Baeuerle and Baltimore, 1996; Ghosh et al., 1998). NF-kB is normally sequestered in the cytoplasm via their non-covalent interaction with a family of inhibitory proteins termed the IkBs (Ghosh et al., 1998). NF-kB signaling is activated by a variety of stimuli such as cytokines and some growth factors, which eventually lead to activation of IkB kinase complex (IKK) (Ghosh et al., 1998; Israel, 2000; Karin, 1999).

IKK in turn phosphorylates IkB resulting in its degradation via the ubiquitin-mediated proteolytic pathway (DiDonato et al., 1997; Ghosh and Karin, 2002; Karin, 1999; Karin et al., 2002; Mercurio et al., 1997; Regnier et al., 1997; Yamaoka et al., 1998; Zandi et al., 1997). This allows the NF-kB complex to translocate into the nucleus, where it engages cognate kB enhancer elements and modulates gene expression. A major negative feedback mechanism to downregulate the activated NF-kB is the transactivation of the IkBα gene by NF-kB (Beg et al., 1993; Brown et al., 1993; Chiao et al., 1994; Sun et al., 1993). Newly synthesized IkBα shuttles between the cytoplasm and the nucleus and can remove NF-kB from the promoters, thus promoting the return of the NF-kB-IkBα complex to the cytoplasm (Arenzana-Seisdedos et al., 1995; Arenzana-Seisdedos et al., 1997; Ghosh and Karin, 2002; Karin et al., 2002). These events result in the termination of the NF-kB transcriptional response (Arenzana-Seisdedos et al., 1995; Arenzana-Seisdedos et al., 1997).

Although NF-kB has a well established function in both immunity and inflammation, recently, it has recently been widely reported that deregulation of NF-kB signaling is associated with oncogenesis and cancer malignancies (Baldwin, 2001; Karin et al., 2002). NF-kB is constitutively active in many human cancers such as breast cancer (Baldwin, 2001; Karin et al., 2002; Nakshatri et al., 1997; Nakshatri and Goulet, 2002; Sovak et al., 1997; Wang et al., 1999).

Furthermore, activated NF-kB in cancer cells has been shown to increase the expression of many genes involved in cell proliferation, metastasis, angiogenesis and anti-apoptosis (Baldwin, 2001; Karin et al., 2002; Nakshatri and Goulet, 2002). Moreover, NF-kB activation has been shown to correlate with higher malignancies and poor prognoses (Baldwin, 2001; Karin et al., 2002; Lessard et al., 2003; Wang et al., 1999). It has been suggested that rapid turnover or degradation of IkBα may be responsible for the constitutive activation of NF-kB in cancer cells (Miyamoto et al., 1994), probably due to constitutive activation of IKK through overexpression of IL-1a, c-myc, EGF and heregulin (Arlt et al., 2002; Bhat-Nakshatri et al., 1998; Bhat-Nakshatri et al., 2002; Nakshatri and Goulet, 2002). However, IkBα protein levels are also elevated in many cancer tissues and cells, that contain constitutively active NF-kB (Nakshatri and Goulet, 2002; Wang et al., 1999), suggesting that the inhibition of NF-kB via IkBα might be disrupted.

Therefore, following the nuclear import, the function of NF-kB, especially the binding between NF-kB and IkBa, might be normally subjected to further regulation and such regulatory mechanisms may be disrupted in cancer cells. Indeed, phosphorylation of nuclear NF-kB by several kinases, such as PKA has been reported to increase transcriptional activity of NF-kB (Zhong et al., 1997; Zhong et al., 1998). However, most of these modifications regulate the transcriptional activity of NF-kB, but not its nuclear localization or turnover. Chen et al. recently reported that the acetylation status of p65 affects its binding affinity to IkBα (Chen et al., 2001; Chen et al., 2002). In this model, the acetylated p65 in the nucleus is refractory to association with IkBα and the deacetylation of p65 by HDAC3 may release its resistance (Chen et al., 2001; Chen et al., 2002). However, since endogenous levels of acetylated p65 have been reported to be quite low in physiological conditions (Chen et al., 2001; Ghosh and Karin, 2002), the biological role of p65 acetylation and its involvement in the constitutive activation of NF-kB in cancer remain to be fully elucidated. Therefore, it is critical to elucidate the regulatory mechanisms other interaction between activated NF-kB and IkBα in the nucleus and its deregulation in cancer. This is important not only for understanding NF-kB-mediated oncogenesis, but also may help in the design of new anti-cancer therapies.

Pin1 is a peptidyl-prolyl isomerase that binds to specific motif of phosphorylated serine or threonine residues that precede proline (pSer/Thr-Pro) in a subset of proteins. This binding induces conformational changes through cis/trans isomerization of these specific pSer/Thr-Pro motifs (Lu et al., 1996; Shen et al., 1998; Yaffe et al., 1997). Since cis and trans pSer/Thr-Pro moieties exist the two completely distinct cis and trans conformations, Pin1-induced conformational changes have been shown to have profound effects on the function of many substrates (Lu et al., 1996; Lu et al., 1999; Ranganathan et al., 1997; Shen et al., 1998; Yaffe et al., 1997; Zhou et al., 1999). This novel “post-phosphorylation” mechanism regulates protein function of Pin1 substrates by modulating activity levels, phosphorylation status, protein-protein interactions, subcellular localization and stability (Lu et al., 2002; Ryo et al., 2003). Pin1 has been shown to be involved in the regulation of many cellular events, such as cell cycle progression, transcriptional regulation and cell proliferation (Lu et al., 2002; Ryo et al., 2003).

Furthermore, Pin1 is highly overexpressed in many human cancers, including breast and prostate cancers and high Pin1 levels correlates with higher malignancy and poor prognosis (Ryo et al., 2002; Ryo et al., 2001; Wulf et al., 2001). Moreover, Pin1 activates several oncogenic pathways such as Neu/Ras/c-Jun and Wnt/β-catenin pathways (Ryo et al., 2002; Ryo et al., 2001; Wulf et al., 2001). Interestingly, Pin1 has been shown to regulate the function of several transcriptional regulators, such as β-catenin, CF2 and p53 by modulating protein stability and subcellular localization (Hsu et al., 2001; Ryo et al., 2001; Wulf et al., 2002; Zacchi et al., 2002; Zheng et al., 2002).

### SUMMARY OF THE INVENTION

The instant invention is based on the discovery that NF-κB function is regulated by Pin1-mediated prolyl isomerization and ubiquitin-mediated proteolysis of p65/RelA. Pin1 binds to the pThr254-Pro motif in p65 and enhances NF-κB activity by inhibiting p65 binding to IkBα and increasing the nuclear accumulation and protein stability of p65. Consequently, Pin1-deficient mice and cells are refractory to NF-κB activation by cytokine signals. Moreover, the p65 mutant (T254A) that cannot act as a Pin1 substrate is both extremely unstable and also fails to transactivate NF-κB target genes. Significantly, p65 stability is controlled by ubiquitin-mediated proteolysis that is facilitated by a cytokine signal inhibitor, SOCS-1 as an ubiquitin ligase. These findings uncover previously unrecognized mechanisms in the control of NF-κB signaling and suggest that their deregulation can offer new insights into the constitutive activation of NF-κB in human diseases such as cancers.

The role of the NF-κB family of proteins in immune, inflammatory, and apoptotic responses is well documented Rayet, B. et al. (1999). Oncogene 18, 6938-6947, Ebralidze, A., et al. (1989). Genes Dev. 3, 1086-1093 and Baeurle, P. A. et al. (1996). Cell 87, 13-20.

Accordingly, The instant invention provides a method of modulating the activity of a NF-kB polypeptide in a cell, comprising contacting the cell with substance that modulates the activity of Pin1 such that the activity of NF-kB is regulated.

In a related embodiment, the activity of NF-kB is the ability to interact with IkBα. In a further embodiment the activity of Pin1 is the peptidyl prolyl isomerase activity. In another related embodiment, the composition that modulates Pin1 is Pin1 modulator., e.g., peptide, a peptide mimetic, a small molecule, or an antibody. The antibody can be a monoclonal or polyclonal antibody. The monoclonal antibody can be humanized, human, or chimeric.

In another embodiment the invention provides a method of inhibiting the isomerization of the pThr254-Pro bond of the P65 subunit of NF-kB the method comprising inhibiting the activity of Pin1. In a related embodiment, the Pin1 activity is inhibited by contacting the Pin1 polypeptide with a compound that binds to the Pin1 active site. In a related embodiment, the compound that binds to the Pin1 active site can be a small molecule, a peptide, or a peptide mimetic. In another related embodiment, the Pin1 activity is inhibited by contacting the Pin1 polypeptide with a compound that binds to the WW domain of Pin1. In a related embodiment, the compound that binds to the WW domain of Pin1 can be a small molecule, a peptide, a phosphoserine peptide or a peptide mimetic.

In anther embodiment the invention provides a method of inhibiting the isomerization of the pThr254-Pro bond in the P65 subunit of NF-kB the method comprising inhibiting the ability of Pin1 to interact with NF-kB. In a related embodiment, the compound that inhibits the ability of Pin1 to interact with NF-kB can be a small molecule, a peptide, or a peptide mimetic.

In another embodiment the invention provides a method of treating a subject having a NF-kB associated condition comprising administering the subject a Pin1 modulator thereby treating the subject. In particular embodiments, the NF-kB disorder is selected from a group consisting of a cell proliferation disorder, an immune response disorder, inflammation, a cell survival disorder and an oncogenesis disorder.

In another embodiment the invention provides a method of treating a subject suffering from a NF-kB associated condition comprising administering the subject an antibody specific for an epitope comprising amino acid residues 254 and 255 of the p65 subunit of NF-kB, thereby treating the subject.

In another embodiment the invention provides a method of increasing the amount of NF-kB proteolysis comprising the step of inhibiting the production of Pin1 thereby allowing NF-kB to be proteolyzed by the ubiquitin mediated proteolysis pathway. The amount of Pin1 produced can be regulated using siRNA or RNAi.

In another embodiment the invention provides a method of treating a subject suffering from a NF-kB associated disorder comprising administering the subject a compound that stimulates the expression of SOCS-1, thereby inhibiting the degredation of NF-kB.

### BRIEF DESCRIPTION OF THE DRAWINGS

FIG. 1. Pin1 Levels Correlate with NF-kB Activation in Human Breast Cancer Tissues.

(A, B) Correlation between Pin1 and p65 localization in human breast cancers and normal tissues. Tissue sections were immunostained with anti-Pin1 or anti-p65 antibodies and visualized by DAB staining (A). The level of Pin1 expression and localization of p65 were determined in 50 breast cancer and 5 normal breast samples and their correlation analyzed by Sperman rank correlation test (P<0.01) (B).

(C) Inhibition of NF-κB activation and NF-κB DNA binding activity in breast cancer cell lines by downregulation of Pin 1. Two breast cancer cell lines were transfected with Pin1-specific or non-specific siRNA together with a NF-kB-Luc or TK-Luc reporter construct for 48 hrs, followed by assaying luciferase activity and Pin1 protein levels (insets) (C) or assaying NF-κB DNA binding activity by EMSA using NF-κB or OCT1 consensus oligonucleotides (D).

FIG. 2. Pin1 Activates NF-kB Signaling.

(A, B) Modulation of NF-kB activation by Pin1. HeLa cells were transfected with vector, HA-Pin1 or Pin1 AS together with the NF-kB reporter construct for 24 hrs and some samples were subjected to 1 ng/ml of TNF-α treatment for 3 hrs. Cells were harvested and subjected to the luciferase assay and immunoblotting with anti-Pin1 antibody.

(C) Increasing NF-kB DNA-binding activity by Pin1. 293T cells were transfected with either vector control or Pin1 for 24 hrs and nuclear extracts were isolated. 5 μg nuclear extracts were incubated with 32 P labeled NF-kB binding oligo or its mutant, followed by gel electrophoresis. For supershift experiments, anti-p50 or p65 antibody was added for 20 min before adding labeled oligo DNA.

FIG. 3. Pin1 Binds the pThr254-Pro Motif in p65 and Inhibits the Binding of p65 to IκB.

(A) In vitro interaction of Pin1 and p65, but not p50 or IκBα. Glutathione beads containing GST or GST-Pin1 were incubated with interphase (I) or mitotic (M) HeLa cell extracts and binding proteins were subjected to immunoblotting with various antibodies indicated.

(B) In vivo interaction of endogenous Pin1 and p65. 293T cell lysates were immunoprecipitated with anti-p65 antibody, followed by immunoblot with anti-Pin1 or anti-p65 antibodies.

(C) Phosphorylation-dependent interaction between Pin1 and p65. 293T cells expressing p65 were incubated with or without calf intestinal alkaline phosphatase (CIP) before subjecting to GST pulldown experiments, followed by immunoblotting with an anti-p65 antibody.

(D) Enhancement of Pin1 and p65 binding by TNF-α. 293T cells expressing Xpress-tagged p65 were treated with PBS or TNF-αfor 3 hrs, followed by GST-pull down experiment and immunoblot with anti-Xpress antibody.

(E, F) Specific binding of Pin1 to the pThr254-Pro motif in p65. 293T cells expressing p65 and its truncated mutants (E) or point mutants (F) were subjected to the GST-pulldown assay.

(G) Failure of Pin1 to bind p65-T254A. 293T cells were co-transfected with Pin1 and Xpress-tagged p65 or its T254A mutant. After cells were incubated with 20 μM MG-132 for 12 hr, they were subjected to immunoprecipitation analysis with anti-Xpress antibody, followed by immunoblot with anti-p65 or anti-Pin1 antibodies.

(H) Recognition of p65, but its mutant p65-T254A by a pThr-Pro-specific antibody. After transfection with Xpress-tagged p65 or its T254A mutant for 24 hr, 293 cells were treated with MG-132 for 12 hr and TNF-α for 3 hrs and subjected to immunoprecipitation with anti-Xpress antibody, followed by immunoblot with anti-pThr-Pro-specific antibody or anti-p65 antibodies.

(I, J) Inhibition of the p65-IκBα binding by Pin 1. (I) HeLa cells expressing HA-tagged Pin1 or control vector for 24 h were subjected to immunoprecipitation (IP) with anti-p65 or anti-IκBα antibody, followed by immunoblotting with various antibodies. (J) 293T cells expressing Xpress-p65 were subjected to immunoprecipitation with anti-Xpress antibody and then incubated with ³⁵S-labeled IkBα and different amounts of Pin1 (0, 0.2 and 2.0 mg/ml) for 30 min. After washing, samples were subjected to SDS-PAGE followed by the autoradiography.

FIG. 4. The Pin1-Binding Site Mutant p65-T254A is Extremely Unstable and Fails to Transactivate NF-kB Target Genes. (A) Failure of p65-T254A to transactivate NF-kB target genes. MEFs were co-transfected with Ig-kB luciferase construct and p65 or its mutants, followed by gene reporter assay. (B, C) Comparison of p65 and its mutant protein stability. Xpress-tagged p65 or its mutants were transfected into 293T cells together with Xpress-LacZ for 24 hrs. Cells were treated with cycloheximide and harvested at indicated time points, followed by immunoblotting with anti-Xpress antibody (B) and semi-quantification with Imagequant (C).

FIG. 5. Pin1^{-/-} Cells are Resistant to NF-κB activation by Cytokines in vitro and in vivo.

(A, B) Resistance to cytokines in Pin1^{-/-} MEFs. After transfection with WT or mutant NF-κB reporter construct, Pin1^{-/-} or WT MEFs were incubated for 3 hr with different concentrations of IL-1β (A) or different cytokines (1 ng/ml of IL-1β, 100 ng/ml of LPS, or 1 ng/ml of TNF-α) (B), followed by a gene reporter assay.

(C, D) Lack of p65 nuclear accumulation and IκB feedback upregulation in response to IL-1β in Pin1^{-/-} MEFs. WT and Pin1^{-/-} MEFs are treated with IL-1β (1 ng/ml) for indicated time points, followed by subjecting whole cell lysates to immunoblot with anti-IκBα and tubulin antibodies, or nuclear fractions to immunoblot with anti-p65 antibody (C). MEFs treated with IL-1β for 3 hrs were immunostained with anti-p65 antibody (D).

(E) Unstable p65 in Pin1^{-/-} MEFs. WT and Pin1^{-/-} MEFs were transfected with Xpress-tagged p65 and Xpress-LacZ for 24 hrs and treated with cycloheximide for the times indicated, followed by immunoblot with anti-Xpress antibody.

(F) Inactive NF-kB in Pin1^{-/-} mammary glands. Mammary glands from WT and Pin1^{-/-} mice (1 day after delivery) were stained with H&E or anti-p65 antibody.

(G-I) Reduced NF-kB activation and increased apoptosis in response to TNF-α in Pin1^{-/-} livers. WT or Pin1^{-/-} mice were injected with 40 mg/kg of recombinant murine TNF-α and killed 3 hours after the injection, followed by subjecting liver sections to immunohistochemistry with anti-p65 antibody or TUNEL staining (G) or subjecting liver lysates to immunoblot with anti-cleaved caspase-3 antibody (H) or a fluorogenic cacpase-3 activity assay in the presence or absence of the inhibitor DEVD-CHO. Data are shown as Mean±SD in 3 independent experiments.

FIG. 6. Poly-ubiquitination of p65 in vitro and in vivo

(A, B) Stabilization of p65-T254A by a proteosome inhibitor. 293T cells expressing Xpress-p65-T254A or Xpress-LacZ were treated with cycloheximide and MG-132 (50 mM) or the solvent DMSO for the times indicated, followed by immunoblot with anti-Xpress antibody (A), followed by semi-quantification with Imagequant (B). (C) Ubiquitination of p65 in vitro. In vitro translated 35 S-labeled p65 was incubated with ubiquitin in the presence or absence of E1 and UbcH5a for times indicated, followed by separation on SDS-PAGE and autoradiography. (D) Ubiquitination of the GST-p65 fragment B, but A or C. GST-p65 truncation mutants (FIG. 3D) were incubated with HeLa S-100 extracts, ubiquitin, E1 and either UbcH5a or UbcH6 for 3 hrs, followed by GST pulldown and immunoblotting with anti-ubiquitin antibody. (E) Ubiquitination of GST-p65 fragment B via UbcH5a. GST-p65-truncated mutant B was subjected to ubiquitination assay in vitro using different E2 enzymes.

(F) Ubiquitination of p65 in vivo. HeLa cells expressing p65, UbcH5a and His-tagged ubiquitin or vector controls were treated with MG-132 or DMSO control for 16 hr, followed by lysis using sonication in a buffer containing 6M urea. Ubiquitin-conjugated proteins were captured with Ni-0.42 Agarose beads and subjected to immunoblot with anti-p65 antibody.

FIG. 7. p65 Binds SOCS-1 in vitro and in vivo

(A) Identification of SOCS-1 in a p65-binding protein. MEFs expressing Xpress-His-doubly tagged p65 fragment B were treated with LPS (100 ng/ml) for 3 hr and then subjected to the Ni-agarose affinity chromatography, followed by immunoprecipitation with anti-Xpress antibody. After silver staining, the bands were excised and subjected to mass spectrometer analysis.

(B) SOCS-1 binding to p65 fragment B in vitro. 293T cells expressing Xpress-tagged p65 or truncation mutants (FIG. 3D) were subjected to GST-pulldown assay with GST or GST-SOCS-1 and immunoblotting with anti-Xpress antibody.

(C) Interaction of expressed p65 and SOCS-1 in vivo. 293T cell were co-transfected with Xpress-p65 and Myc-SOCS-1, followed by immunoprecipitation with control IgG, anti-Xpress or anti-Myc antibodies, followed by immunoblot with indicated antibodies.

(D) Interaction of endogenous p65 and SOCS-1 in vivo. Mouse primary spelenocytes were incubated with or without LPS for 4 hr and then subjected to immunoprecipitation with anti-p65 antibody, followed by immunoblot with anti-SOCS-1 antibody.

FIG. 8. SOCS-1 Modulates Ubiquitination and Protein Stability of p65

(A, B) SOCS-1 inhibition of NF-κB activation by IL-1β (A) or p65 (B). 293T cells stably expressing IL-IR were co-transfected with control vector, SOCS-1 or SOCS-1ΔS and either WT or mutant NF-κB luciferase construct, followed by IL-1β (2 ng/ml) treatment and gene reporter assay (A). MEFs were co-transfected with either WT or mutant NF-kB luciferase construct, and either control vector, SOCS-1 or SOCS-1ΔS and p65, followed by gene reporter assay (B).

(C) SOCS-1 modulation of p65, but not p50 levels. HeLa cells were transfected with vector, SOCS-1 or SOCS-1ΔS, followed by immunoblot with anti-p65, p50 and SOCS-1 antibodies.

(D) SOCS-1 modulation of p65 protein stability. 293T cells were co-transfected with Xpress-p65, Xpress-LacZ and SOCS-1, SOCS-1+Pin1, SOCS-1ΔS or vector and then treated with cycloheximide (100 μg/ml), followed by immunoblotting analysis with anti-Xpress antibody (left panels) and semi-quantification (right panel).

(E) SOCS-1 modulation of p65 ubiquitination in vitro. GST-p65 fragment B was subjected to an in vitro ubiquitination reaction in the presence or absence of cell lysates from 293T cells transfected with SOCS-1, SOCS-1ΔS, or a control vector, followed by GST pulldown and immunoblot with anti-ubiquitin antibody.

(F) SOCS-1 modulation of p65 ubiquitination in vivo. HeLa cells were transfected with Xpress-p65, His-tagged ubiquitin and SOCS-1, SOCS-1ΔS or control vector for 24 hr and then treated with MG-132 for 16 h and ubiquitinated proteins were captured by Ni beads, followed by immunoblotting with anti-Xpress antibody.

(G) Pin1 blocks the SOCS-1 induced ubiquitination of p65. HeLa cells were transfected with Xpress-p65, His-tagged ubiquitin, UbcH5a and either control vector, SOCS-1 or SOCS-1 plus Pin1 for 24 hr and then treated with MG-132 and MG-115 for 16 h and ubiquitinated proteins were captured by Ni beads, followed by immunoblotting with anti-p65 polyclonal antibody.

(H, I) p65 is less ubiquitinated and more stable in SOCS-1^{-/-} cells. (H) WT or SOCS-1^{-/-} MEFs were transfected with Xpress-p65, His-tagged ubiquitin and UbcH5a for 24 hr, followed by. ubiquitination assay, as described in G. (I) After WT or SOCS-1^{-/-} MEFs are transfected with both Xpress-p65 and Xpress-LacZ for 24 hrs, they were treated with cycloheximide, followed by immunobloting analysis with anti-Xpress antibody (upper) and semi-quantification (lower).

FIG. 9. Schematic Model of Two Step NF-κB Regulation by Pin1 and SOCS-1 NF-kB signaling is activated by IKK-mediated phosphorylation and subsequent degradation of IkBa, which results in the translocation of NF-kB into the nucleus. Our results reveal that nuclear p65 is further regulated by Pin1-catalyzed prolyl isomerization and ubiquitin-mediated proteolysis. Pin1 targets to the pThr254-Pro motif in p65 and inhibits its binding with IkBa, enhancing the nuclear accumulation and protein stability of p65 and transcriptional activity of NF-kB. Furthermore, when p65 is exported into the cytoplasm, it is regulated by ubiquitin-mediated proteolysis via UbcH5a and SOCS-1. Overexpression of Pin1 and/or downregulation of SOCS-1 contribute to the constitutive activation of NF-kB in cancer.

FIG. 10. Pin1 Activates NF-kB Independently on IkB Phosphorylation.

(A, B) HeLa cells transfected with vector or Pin1 were subjected to immunoblotting with anti-phospho IkBa (Ser32), IkBa and tubulin antibodies (A), or immunoprecipitation with anti-IKKα antibody, followed by the in vitro kinase assay using GST-IkBα as a substrate (B).

(C, D) IKK1/IKK2 double knockout or NEMO−/− MEFs were transfected with Pin1 or vector and Ig-kB luciferase construct (C) or with Pin1, Ig-kB luciferase construct and p65 or p50, followed by gene reporter assay.

FIG. 11. The Ribbon diagram of the NF-kB and IkBα Complex and the Pin1 interaction with p65.

(A, B) Ribbon diagram of the NF-kB (p65, green; p50, gray) and IkBα (pink) complex are shown in upper panels, and some binding interface between IkBα and p65 is highlighted in the lower panels. When p65 binds to IkBa, Arg253 in p65 is exposed and may form some hydrogen bonds with IkBα residues, as reported (Huxford et al., 1998; Jacobs and Harrison, 1998). In this situation, Thr254 is buried within the complex (A). However, when NF-kB is released from IkBa, a long loop five including Arg253 and Thr254 becomes flexible and Thr254 is exposed. When Thr254 is phosphorylated, Pin1 binds and isomerizes the pThr254-Pro motif in p65, which would disrupt the IkBα binding surface and thereby inhibit the binding of p65 to IkBα (B). However, this would not affect the interaction between p65 and p50 based on the structure.

### DETAILED DESCRIPTION

The studies presented herein identified two novel regulatory mechanisms to control NF-kB (Accession number: NP_{—}003989) signaling. It has been shown herein, that Pin1 specifically binds to the pThr254-Pro motif in p65 and enhances its nuclear localization and protein stability likely via inhibiting the p65 binding to IkBα (Accession number: NP_{—}0656390). The biological significance of this Pin1 (Accession number: AAC50492) regulation of p65 was further confirmed by the findings that Pin1-deficient cells are refractory to NF-kB activation by cytokine signals due to rapid p65 nuclear export and degradation, and that a p65-T254A mutant that cannot act as a Pin1 substrate is extremely unstable and fails to transactivate NF-kB target genes. Consistent with these findings, it has been further demonstrated that p65 protein stability is regulated by ubiquitin-mediated proteolysis and that the cytokine signal inhibitor SOCS-1 is a putative p65 ubiquitin ligase. Moreover, SOCS-1 plays a crucial role in regulating p65 ubiquitination and protein stability. These results demonstrate for the first time that NF-kB is regulated by Pin 1-catalyzed prolyl isomerization and ubiquitin-mediated proteolysis of p65.

Given that the upregulation of Pin1 and downregulation of SOCS-1 is evident in many human cancers, deregulation of these new mechanisms likely contribute to the constitutive activation of NF-kB in cancers. By binding and isomerizing specific pSer/Thr-Pro bonds, Pin1 regulates the conformation and function of specific phosphorylated proteins and thus may play an important role in gene expression, cell cycle regulation and oncogenesis (Lu et al., 2002; Ryo et al., 2003). It has been demonstrated herein that Pin1 activates NF-kB signaling without affecting IκK activity and IkBα phosphorylation. Furthermore, Pin1 directly binds to the Thr254-Pro motif in p65. This site is located near the “hot spots” for the interaction of p65 and IkBα. Based on the crystal structure of the NF-kB-IkBa complex, the binding of IkBα to p65 strikingly stimulates the conformational changes of p65 around the loop 5 region including Ser238-Asp243 and Arg253, all of which have been reported to play important roles in the p65 binding to IkBα (Huxford et al., 1998; Jacobs and Harrison, 1998).

The Thr254-Pro motif is buried inside in the complex. When IkBα is degraded by upstream signaling and NF-kB is released from IkBa., the dimerization domain of p65 becomes more flexible and the Thr254-Pro motif can be exposed and subjected to the phosphorylation. This phosphorylation newly creates a Pin1 binding site. Subsequently, Pin1 binds and isomerases the pThr254-Pro motif, which would completely disrupt the binding interface of p65 for IkBα. However, based on the crystal structure and the current model, the binding between Pin1 and IkBα may not affect the interaction of p50 and p65 heterodimerization. Consistent with this possibility, it was found that Pin1 inhibits the association of p65 with IkBa, but not with p50, as detected by co-immunoprecipitation and in vitro binding assays. Furthermore, Pin1 overexpression inhibits, but disruption of Pin1 enhances the nuclear export and subsequent degradation of p65. Importantly, the Pin1-binding site mutant p65-T254A was extremely unstable and failed to transactivate NF-kB down-stream genes. This striking functional change following a single amino acid substitution further supports the importance of the phosphorylation and subsequent Pin1 interaction at this site for the proper NF-kB regulation. These results indicate that Pin1 plays a critical role in enhancing the stability, nuclear localization and transcriptional activity of p65. This is consistent with the previous findings that Pin1 regulates the stability and nuclear localization of several other proteins such as β-catenin, p53, cyclin D1 and CF1, although the underlying mechanisms vary depending on the substrates (Hsu et al., 2001; Liou et al., 2002; Ryo et al., 2001; Wulf et al., 2002; Zacchi et al., 2002; Zheng et al., 2002). For example, in the case of p53, Pin1 increases p53 protein stability and transcriptional activity likely via inhibiting its binding to MDM2 (Wulf et al., 2002; Zacchi et al., 2002; Zheng et al., 2002).

In the case of β-catenin, Pin1 inhibits the β-catenin binding to APC and increases its nuclear translocation, protein stability and transcriptional activity, as is the case for p65 (Ryo et al., 2001). Further studies are needed to identify upstream kinases that phosphorylate the Thr254-Pro motif in p65 and their function and regulation.

The ability of Pin1 to regulate the protein stability of p65 led to another surprising finding in this study, which is the ubiquitin-mediated proteolysis of p65. Although the ubiquitin-mediated proteolysis of IkBα has been well characterized (Baeuerle and Baltimore, 1996; Ghosh et al., 1998), a similar regulation has not been previously described for NF-kB itself. Although p65 is quite stable in WT MEFs and other cells expressing Pin1, it became extremely unstable in Pin1−/− MEFs, but could be stabilized by the proteasome inhibitor MG-132. Furthermore, even in Pin1+/+ cells, the point mutation in p65 (T254A) that disrupts its binding to Pin1 converts p65 from a stable into an extremely unstable protein due to rapid nuclear export and subsequent protein degradation. These results indicate that p65 is highly unstable intrinsically and regulated normally.

The regulation of p65 protein stability has been further supported by our findings that p65 is poly-ubiquitinated in vitro and in vivo, which is enhanced by UbcH5a, but none of the other ubiquitin conjugating E2 enzymes examined. Furthermore, it has been demonstrated that the putative p65 ubiquitin ligase to be SOCS-1. SOCS-1 directly interacts with p65 and enhances its ubiquitination and degradation, inhibiting NF-kB activation by cytokines. Significantly, SOCS-1 is a member of suppressors of cytokine signaling (SOCS) family of proteins, and has been also shown to promote the ubiquitination and degradation of JAK2 and Vav (De Sepulveda et al., 2000; Frantsve et al., 2001; Kamizono et al., 2001; Kile et al., 2002). SOCS-1 is a putative tumor suppressor that is able to inhibit cell proliferation induced by a constitutively active form of the KIT receptor, TEL-JAK2 and v-ABL, as well as to reduce the metastasis of BCR-ABL transformed cells (Kile and Alexander, 2001; Rottapel et al., 2002; Yoshikawa et al., 2001).

Recently, it was reported that SOCS-1 inhibits LPS-induced macrophage activation (Kinjyoet al., 2002; Nakagawa et al., 2002). In these cases, it has been shown that LPS induces SOCS1, which then negatively regulate LPS signaling. SOCS1−/− macrophages exhibit the up-regulation of LPS-induced IkBa phosphorylation and NF-kB activation. These previous studies have shown that SOCS-1 suppressed NF-kB activation by LPS by inhibiting the upstream signaling pathway for IkBα phosphorylation, although detailed molecular mechanism has not been described. In the current study, we have shown that SOCS-1 can directly target p65 and enhance its ubiquitin-mediated proteolysis, resulting in the downregulation of NF-kB. Therefore, it is possible that SOCS-1 can downregulate NF-kB signaling through multiple mechanisms.

The biological significance of the regulation of NF-kB by Pin1 and SOCS-1 has also been revealed by mammary gland phenotypes in mouse models. The importance of NF-kB signaling for mammary gland development during late pregnancy and precocious lactation has been reported (Brantley et al., 2001; Cao et al., 2001; Clarkson, 2002; Fata et al., 2000; Geymayer and Doppler, 2000; Hennighausen and Robinson, 2001). Although p65 knockout mice are embryonic lethal (Beg et al., 1995), IKKα knockout mice clearly exhibit a severe impairment of mammary gland development during and after pregnancy (Cao et al., 2001). Likewise, in Pin1 knockout mammary glands, NF-kB is not active and the epithelial cells fail to undergo the massive proliferative changes during pregnancy (Liou et al., 2002). In contrast, SOCS-1 deficient mice exhibit accelerated mammary gland development (Lindeman et al., 2001). These results further support the functional connection of Pin1 and SOCS-1 with NF-kB signaling in vivo.

Significantly, deregulation of Pin1-catalyzed prolyl isomerization and ubiquitin-mediated proteolysis of p65 may offer new insights into constitutive activation of NF-kB in many human cancers. It has been demonstrated that Pin1 is highly overexpressed in many human cancers (Ryo et al., 2003; Ryo et al., 2002; Ryo et al., 2001; Wulf et al., 2001), whereas the SOCS-1 gene is silenced in many human malignancies (Rottapel et al., 2002; Yoshikawa et al., 2001). Because overexpression of Pin1 reduces nuclear export of p65 likely via inhibiting its binding to the nuclear-cytoplasmic shuttling protein IkBa, NF-kB would be accumulated in the nucleus and be constitutively active. Additionally, if some p65 proteins is exported into the cytoplasm by the interaction with newly synthesized IkBα or other exporters, it might not be degraded properly via the ubiquitin-proteasome pathway because of the downregulation of SOCS-1. Cytoplasmic NF-kB can again translocate into the nucleus due to the phosphorylation and subsequent degradation of IkBα by IKK, which is activated by upstream oncogenic signals.

Under these conditions, since negative feedback mechanisms that downregulate NF-kB would be disrupted, NF-kB would become constitutively activated in the nucleus and thus activate downstream genes even though IkBα is elevated. Consistent with this notion, Pin1 levels correlate with NF-kB activation in human breast cancer tissues and inhibition of Pin1 suppresses NF-kB activation in breast cancer cells. Furthermore, this model can also provide an explanation as to why NF-kB is constitutively active even though IkBα is also elevated in cancer tissues. Thus, the instant results suggest that Pin1-dependent prolyl isomerization and ubiquitin-mediated proteolysis of p65 may be novel mechanisms that regulate NF-kB signaling and their deregulation may play a critical role in constitutive activation of NF-kB during and after oncogenesis.

Accordingly, the instant invention provides methods of modulating NF-kB by modulating the activity and/or expression of Pin1. The invention further provides methods of treating a subject suffering from an NF-kB associated disease or disorder.

The term “NF-kB associated disease” or “NF-kB associated disorder” is intended to include diseases and disorders in which abberant expression, degredation or activity of NF-kB leads to a physiological result that is undesired. In particular embodiments, the disease or disorder is a cell proliferative disorder, e.g., cancer, immune response disorders and inflammatory disorders.

The term “cell proliferative disorder” is intended to include diseases and disorders characterized by abnormal cell growth. Included in these diseases and disorders are carcinomas, sarcomas, mylomas, and neoplasias. Exemplary types of cell proliferative disorders include As used herein the term “cell proliferative disorder” includes diseases and disorders such as oligodendroglioma, astrocytoma, glioblastomamultiforme, cervical carcinoma, endometriod carcinoma, endometrium serous carcenoma, ovary endometroid cancer, ovary Brenner tumor, ovary mucinous cancer, ovary serous cancer, uterus carcinosarcoma, breast cancer, breast lobular cancer, breast ductal cancer, breast medullary cancer, breast mucinous cancer, breast tubular cancer, thyroid adenocarcinoma, thyroid follicular cancer, thyroid medullary cancer, thyroid papillary carcinoma, parathyroid adenocarcinoma, adrenal gland adenoma, adrenal gland cancer, pheochromocytoma, colon adenoma mild displasia, colon adenoma moderate displasia, colon adenoma severe displasia, colon adenocarcinoma, esophagus adenocarcinoma, hepatocelluar carcinoma, mouth cancer, gall bladder adenocarcinoma, pancreatic adenocarcinoma, small intestine adenocarcinoma, stomach diffuse adenocarcinoma, prostate (hormone-refract), prostate (untreated), kidney chromophobic carcinoma, kidney clear cell carcinoma, kidney oncocytoma, kideny papillary carcinoma, testis non-seminomatous cancer, testis seminoma, urinary bladder transitional carcinoma, lung adenocarcinoma, lung large cell cancer, lung small cell cancer, lung squmous cell carcinoma, Hodgkin lymphoma, MALT lymphoma, non-hodgkins lymphoma (NHL) diffuse large B, NHL, thymoma, skin malignant melanoma, skin basolioma, skin squamous cell cancer, skin merkel zell cancer, skin benign nevus, lipoma, and liposarcoma.

The term “immune response disorder” is intended to include immune disorders in which there is aberrant expression or regulation of NFκB that leads to a increased or decreased immune response by an individual. For example, diseases and disorders such as autoimmune disease, dermatosis, posriasis, dennatitis, tissue and organ rejection are intended to be included in the instant invention.

The term “inflammatory disorder” is intended to include diseases and disorders in which there is aberrant expression or regulation of NFκB. Further, “inflammatory disorder” is intended to include a disease or disorder characterized by, caused by, resulting from, or becoming affected by inflammation. An inflammatory disorder may be caused by or be associated with biological and pathological processes associated with, for example, NF-kB mediated processes. Examples of inflammatory diseases or disorders include, but are not limited to, acute and chronic inflammatory disorders such as asthma, psoriasis, rheumatoid arthritis, osteoarthritis, psoriatic arthritis, inflammatory bowel disease (Crohn's disease, ulcerative colitis), ankylosing spondylitis, sepsis, vasculitis, and bursitis; autoimmune diseases such as Lupus, Polymyalgia, Rheumatica, Scleroderma, Wegener's granulomatosis, temporal arteritis, cryoglobulinemia, and multiple sclerosis; transplant rejection; osteoporosis; cancer, including solid tumors (e.g., lung, CNS, colon, kidney, and pancreas); Alzheimer's disease; atherosclerosis; viral (e.g., HIV or influenza) infections; chronic viral (e.g., Epstein-Barr, cytomegalovirus, herpes simplex virus) infection; and ataxia telangiectasia.

In preferred embodiments, the instant invention provides method of treating conditions in which NF-κB is know to be involved in, e.g., inflammatory disorders; particularly rheumatoid arthritis, inflammatory bowel disease, and asthma; dermatosis, including psoriasis and atopic dennatitis; autoimmune diseases; tissue and organ rejection; Alzheimer's disease; stroke; atherosclerosis; restenosis; cancer, including Hodgkins disease; and certain viral infections, including AIDS; osteoarthritis; osteoporosis; and Ataxia Telangiestasia.

Modulators of Pin1

Exemplary peptide and peptide mimetic modulators of Pin1 are described in U.S. Pat. No. 6,462,173, issued Oct. 8, 2002. Exemplary, small molecule modulators of Pin1 activity are described in U.S. Pat. No. 6,462,173, WO 03074550 A2, WO 03073999 A2, WO 03074497 A1, WO 04028535A1, WO 03074001A2, WO 03074002A2, and U.S. Provisional Application No. 60/537,171, filed Jan. 16, 2004, entitled “Pin1-Modulating Compounds and Methods of Use Thereof.” Modulators of Pin1 can further be identified by methods known in the art.

Methods of designing modulators of Pin1 polypeptides are described, for example, in WO 03074001 A2.

Modulators of Pin1 can further be antibodies that recognize Pin1. These antibodies can be monoclonal or polyclonal antibodies and can modulate Pin1 activity, e.g., the ability of Pin1 to interact with NF-kB, by blocking interaction with a target molecule. Antibodies of the invention are further described herein.

Preferred epitopes encompassed by the antigenic peptide are regions of Pin1 or p65 subunit of NF-kB that are located on the surface of the protein, e.g., hydrophilic regions, as well as regions with high antigenicity. Even more preferred antibodies are those that recognize epitopes that contain residues that comprise part of the site of interaction between Pin1 and NF-kB.

A Pin1 or NF-kB immunogen typically is used to prepare antibodies by immunizing a suitable subject, (e.g., rabbit, goat, mouse or other mammal) with the immunogen. An appropriate immunogenic preparation can contain, for example, recombinantly expressed A Pin1 or NF-kB protein or a chemically synthesized Pin1 or NF-kB polypeptide. The preparation can further include an adjuvant, such as Freund's complete or incomplete adjuvant, or similar immunostimulatory agent. Immunization of a suitable subject with an immunogenic a Pin1 or NF-kB preparation induces a polyclonal anti-Pin1 or anti-NF-kB antibody response.

Accordingly, another aspect of the invention pertains to anti-Pin1 or anti-NF-kB antibodies. The term “antibody” as used herein refers to immunoglobulin molecules and immunologically active portions of immunoglobulin molecules, i.e., molecules that contain an antigen binding site which specifically binds (immunoreacts with) an antigen, such as Pin1 or NF-kB. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind PCIP. The term “monoclonal antibody” or “monoclonal antibody composition”, as used herein, refers to a population of antibody molecules that contain only one species of an antigen binding site capable of immunoreacting with a particular epitope of Pin1 or NF-kB. A monoclonal antibody composition thus typically displays a single binding affinity for a particular Pin1 or NF-kB protein with which it immunoreacts. Antibodies to Pin1 are described in U.S. Pat. No. 6,596,848, the entire contents of which are expressly incorporated by reference.

Polyclonal anti-Pin1 or anti-NF-kB antibodies can be prepared as described above by immunizing a suitable subject with a PCIP immunogen. The anti-Pin1 or anti-NF-kB antibody titer in the immunized subject can be monitored over time by standard techniques, such as with an enzyme linked immunosorbent assay (ELISA) using immobilized PCIP. If desired, the antibody molecules directed against Pin1 or NF-kB can be isolated from the mammal (e.g., from the blood) and further purified by well known techniques, such as protein A chromatography to obtain the IgG fraction. At an appropriate time after immunization, e.g., when the anti-Pin1 or anti-NF-kB antibody titers are highest, antibody-producing cells can be obtained from the subject and used to prepare monoclonal antibodies by standard techniques, such as the hybridoma technique originally described by Kohler and Milstein (1975) Nature 256: 495-497) (see also, Brown et al. (1981) J. Immunol. 127: 539-46; Brown et al. (1980) J. Biol. Chem 0.255: 4980-83; Yeh et al. (1976) Proc. Natl. Acad. Sci. USA 76: 2927-31; and Yeh et al. (1982) Int. J. Cancer 29: 269-75), the more recent human B cell hybridoma technique (Kozbor et al. (1983) Immunol Today 4: 72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54: 387-402; M. L. Gefter et al. (1977) Somatic Cell Genet. 3: 231-36). Briefly, an immortal cell line (typically a myeloma) is fused to lymphocytes (typically splenocytes) from a mammal immunized with a PCIP immunogen as described above, and the culture supernatants of the resulting hybridoma cells are screened to identify a hybridoma producing a monoclonal antibody that binds Pin1 or NF-kB.

Any of the many well known protocols used for fusing lymphocytes and immortalized cell lines can be applied for the purpose of generating an anti-Pin1 or anti-NF-kB monoclonal antibody (see, e.g., G. Galfre et al. (1977) Nature 266: 55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind Pin1 or NF-kB, e.g., using a standard ELISA assay.

Alternative to preparing monoclonal antibody-secreting hybridomas, a monoclonal anti-PCIP antibody can be identified and isolated by screening a recombinant combinatorial immunoglobulin library (e.g., an antibody phage display library) with Pin1 or NF-kB to thereby isolate immunoglobulin library members that bind anti-Pin1 or anti-NF-kB. Kits for generating and screening phage display libraries are commercially available (e.g., the Pharmacia Recombinant Phage Antibody System, Catalog No. 27-9400-01; and the Stratagene Surf/ZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9: 1370-1372; Hay et al. (1992) Hum. Antibody. Hybridomas 3: 81-85; Huse et al. (1989) Science 246: 1275-1281; Griffiths et al. (1993) EMBO J. 12: 725-734; Hawkins et al. (1992) J. Mol. Biol. 226: 889-896; Clarkson et al. (1991) Nature 352: 624-628; Gram et al. (1992) Proc. Natl. Acad. Sci. USA 89: 3576-3580; Garrad et al. (1991) Bio/Technology 9: 1373-1377; Hoogenboom et al. (1991) Nuc. Acid Res. 19: 4133-4137; Barbas et al. (1991) Proc. Natl. Acad. Sci. USA 88: 7978-7982; and McCafferty et al. Nature (1990) 348: 552-554.

Additionally, recombinant anti-Pin1 or anti-NF-kB antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240: 1041-1043; Liu et al. (1987) Proc. Natl. Acad. Sci. USA 84: 3439-3443; Liu et al. (1987) J. Immunol. 139: 3521-3526; Sun et al. (1987) Proc. Natl. Acad. Sci. USA 84: 214-218; Nishimura et al. (1987) Canc. Res. 47: 999-1005; Wood et al. (1985) Nature 314: 446-449; and Shaw et al. (1988) J. Natl. Cancer Inst. 80: 1553-1559); Morrison, S. L. (1985) Science 229: 1202-1207; Oi et al. (1986) BioTechniques 4: 214; Winter U.S. Pat. No. 5,225,539; Jones et al. (1986) Nature 321: 552-525; Verhoeyan et al. (1988) Science 239: 1534; and Beidler et al. (1988) J. Immunol. 141: 4053-4060.

An anti-Pin1 or anti-NF-kB antibody (e.g., monoclonal antibody) can be used to isolate Pin1 or NF-kB by standard techniques, such as affinity chromatography or immunoprecipitation. An anti-Pin1 or anti-NF-kB antibody can facilitate the purification of natural PCIP from cells and of recombinantly produced Pin1 or NF-kB expressed in host cells. Moreover, an anti-Pin1 or anti-NF-kB antibody can be used to detect Pin1 or NF-kB protein (e.g., in a cellular lysate or cell supernatant) in order to evaluate the abundance and pattern of expression of the Pin1 or NF-kB protein. Anti-Pin1 or anti-NF-kB antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, -galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Further, modulators of Pin1 can be modulators of Pin1 expression such as antisense RNA, siRNA or RNAi, such that Pin1 polypeptide are never translated. RNAi is a ubiquitous mechanism of gene regulation in plants and animals in which target mRNAs are degraded in a sequence-specific manner as described in Sharp, et al. (2001) Genes Dev. 15, 485-490, Hutvagner, G et al. (2002) Curr. Opin. Genet. Dev. 12, 225-232, Zamore, P. D. et al. (2000) Cell 101, 25-33 and Elbashir, S. M. et al. (2001) Nature 411, 494-498. siRNA technology is described in Elbashir, et al. (2001). Genes Dev. 15, 188-200, Hammond, S. M., et al. Nature (2000) 404, 293-296, and Bernstein, E., et al. (2001). Nature 409, 363-366.

The siRNAs molecules of the invention can comprise 16-30, e.g., 16, 17, 18, 19, 20, 21, 22, 23, 24, 25, 26, 27, 28, 29, or 30 nucleotides in each strand, wherein one of the strands is substantially complementary, e.g., at least 80% complementary (or more, e.g., 85%, 90%, 95%, or 100%)(for example, having 3, 2, 1, or 0 mismatched nucleotide(s)), to a target region. A target region differs by at least one base pair between the wild type and mutant allele, e.g., a target region comprising a gain-of-function mutation, and the other strand is identical or substantially identical to the first strand. The dsRNA molecules of the invention can be chemically synthesized or can be transcribed be in vitro from a DNA template or engineered RNA precursor.

The dsRNA molecules can be designed using any method known in the art, for instance, by using the following protocol:

1. Beginning with an AUG start codon, search for AA dinucleotide sequences; each AA and the 3′ adjacent 16 or more nucleotides are potential siRNA targets. The siRNA should be specific for a target region that differs by at least one base pair between the wild type and mutant allele, e.g., a target region comprising the gain-of-function mutation. In cases where the gain-of-function mutation is associated with one or more other mutations in the same gene, the siRNA can be targeted to any of the mutations. In some cases, the siRNA is targeted to an allelic region that does not comprise a known mutation but does comprise an allelic variation of the wild-type (reference) sequence. The first strand should be complementary to this sequence, and the other strand is identical or substantially identical to the first strand. In one embodiment, the nucleic acid molecules are selected from a region of the target allele sequence beginning at least 50 to 100 nt downstream of the start codon, e.g., of the sequence of SOD1. Further, siRNAs with lower G/C content (35-55%) may be more active than those with G/C content higher than 55%. Thus in one embodiment, the invention includes nucleic acid molecules having 35-55% G/C content. In addition, the strands of the siRNA can be paired in such a way as to have a 3′ overhang of 1 to 4, e.g., 2, nucleotides. Thus in another embodiment, the nucleic acid molecules can have a 3′ overhang of 2 nucleotides, such as TT. The overhanging nucleotides can be either RNA or DNA.

2. Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at www.ncbi.nlm.nih.gov/BLAST.

3. Select one or more sequences that meet your criteria for evaluation. Further general information about the design and use of siRNA may be found in “The siRNA User Guide,” available at http://www.mpibpc.gwdg.de/abteilungen/100/105/sirna.html. The siRNAs of the invention generally have one or more modified bases in the antisense strand, e.g., U(5Br), U(5I), and/or DAP. Such modified siRNAs can be synthesized with the modified base.

Further modulators of Pin1 can be peptides that mimic the natural substrate of Pin1, i.e., a phosphoserine, or phosphothreonine moiety. In a particular embodiment, the peptide can mimic the recognition site of Pin1 on the p65 subunit of NF-κB.

Modulators of NF-κB can be preformed using, for example, a cell based luciferase reporter assay as described in Breton, J. J and Chabot-Fletcher, M. C. JPET, 282, 459-466 (1997). Briefly, U937 human histiocytic lymphoma cell line permanently transfected with the NF-.kappa.B reporter plasmids (see below) are cultured in the above medium with the addition of 250.mu.g/ml Geneticin (G418 sulfate, Life Technologies, Grand Island, N.Y.). The luciferase reporter assay is conducted in the transfected U937 clones. These are twice centrifuged at 300.times.g for 5 min and resuspended in RPMI 1640 with 10% FBS to a density of 1.times.10.sup.6 cells/ml. One ml aliquots are added to the wells of 24-well plates. Compound or dimethyl sulfoxide (DMSO) carrier (1.mu.l) is added to the appropriate wells and the plates are incubated at 37.degree. C., 5% CO.sub.2 for 30 min. The stimulus is added (5 ng/ml TNF.alpha., 100 ng/ml LPS, or 0.1.mu.M PMA) and the samples incubated for 5 hours at 37.degree. C., 5% CO.sub.2, transferred to 1.9 ml polypropylene tubes, and centrifuged at 200.times.g for 5 min. The cell pellets are washed twice in 1 ml PBS without Ca.sup.2+ and Mg.sup.2+, and centrifuged as indicated above. The resulting cell pellets are lysed in 50.mu.l 1.times. lysis buffer (Promega Corporation, Madison, Wis.), vortexed and incubated for 15 min at room temperature. A 20.mu.l aliquot of each lysate is transferred to an opaque white 96-well plate (Wallac Inc., Gaithersburg, Md.) and assayed for luciferase production in a MicroLumat LB 96 P luminometer (EG&G Berthold, Bad Wilbad, Germany). The luminometer dispenses 100.mu.l luciferase assay reagent (Promega Corporation, Madison, Wis.) into each well and the integrated light output is recorded for 20 sec. Light output is measured in relative light units (RLUs).

Further, modulators of the instant invention can be tested for their ability to interact with and or modulate the activity of NF-κB using the in vivo assays described in the examples section herein. Further, the modulators of the invention can be tested in an animal model, e.g., an animal model of NF-κB as described in May, et al. (2000). Science 289, 1550-1553, or the Anti-inflammatory activity in vivo is assessed using the phorbol ester-induced ear inflammation model in mice. Phorbol myristate acetate (PMA) (4.mu.g/20.mu.l acetone) is applied to the inner and outer surfaces of the left ear of Male Balb/c mice (6/group) (Charles River Breeding Laboratories, Wilmington, Mass.). Four hours later, compound dissolved in 25.mu.l acetone is applied to the same ear. The thickness of both ears is measured with a dial micrometer (Mitutoyo, Japan) after 20 hours and a second topical dose of compound is applied. Twenty-four hours later, ear thickness measurements are taken and the data expressed as the change in thickness (.times. 10.sup.-3 cm) between treated and untreated ears. The inflamed left ears are then removed and stored at −70.degree. until assayed for myeloperoxidase (MPO) activity, a measure of inflammatory cell infiltration.

Pharmaceutical Compositions and Administration

The invention encompasses use of the polypeptides, nucleic acids, small molecules, antibodies and other agents in pharmaceutical compositions to administer to the cells which are involved in an NF-kB associated disorder as disclosed herein. The molecules, protein, nucleic acids, and antibodies (also referred to herein as “active compounds”) can be incorporated into pharmaceutical compositions suitable for administration to a subject, e.g., a human. Such compositions typically comprise the nucleic acid molecule, protein, modulator, or antibody and a pharmaceutically acceptable carrier. It is understood however, that administration can also be to cells in vitro as well as to in vivo model systems such as non-human transgenic animals.

The term “administer” is used in its broadest sense and includes any method of introducing the compositions of the present invention into a subject. This includes producing polypeptides or polynucleotides in vivo as by transcription or translation, in vivo, of polynucleotides that have been exogenously introduced into a subject. Thus, polypeptides or nucleic acids produced in the subject from the exogenous compositions are encompassed in the term “administer.”

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions. A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fingi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a small molecule or an antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For oral administration, the agent can be contained in enteric forms to survive the stomach or further coated or mixed to be released in a particular region of the GI tract by known methods. For the purpose of oral therapeutic administration, the active compound can be incorporated with excipients and used in the form of tab lets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. “Dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see e.g., Chen et al. (1994) PNAS 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system. The pharmaceutical compositions can be included in a container, pack, or dispenser together with instructions for administration.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments. In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The entire contents of each of the aforementioned patent applications and references are hereby expressly incorporated herein by reference in their entireties.

The details and features of the instant invention are further demonstrated by the examples presented herein.

### EXAMPLES

The following experimental procedures were used in the Examples set forth herein.

Immunohistochemistry

Breast cancer array was purchased from Immugenex and immunostained as described previously (Ryo et al., 2001). Briefly, slides were deparaffinized in xylen, hydrated 100% and 75% ethanol and then washed with H2O. Antigen recapture procedure was performed by boiling in a microwave for 10 min in 1× antigen retreat citra (Biogene). Slides were treated with PBS containing 5% goat serum and 0.1% Triton X100 for blocking, and then treated with anti-Pin1polyclonal antibody or anti-p65 monoclonal antibody (Chemicon; MAB3026) at 4° C. in humidified chamber for 12 hr. After washing with PBS, slides were incubated with biotinized secondary antibody for 2 hr. Immunohistochemical analysis was performed using Vectastain ABC kit and DAB-staining solution (Vector Laboratories, Burlingame, Calif.).

Gene Reporter Assay

Approximately 60% confluent cells were transfected in triplicate in 12 well dishes with Effectene (Qiagen). Gene reporter assays were performed with Dual-Luciferase reporter assay system (Promega) at 24-36 hr after transfection as described previously (Wulf et al., 2001). pRL-TK (Promega) was used as an internal control for transfection efficiency. All results are expressed as X±SD of independent triplicate cultures. For gene reporter assays in breast cancer cell line, cells were transfected with pSuppressor Neo vector encoding Pin1 specific siRNA (5′-GCCACATCACTAACGCCAGC-3′) or non-specific siRNA (5′-TCGTATGTTGTGTGGAATTG-3′) together with Ig-kB luciferase construct and pRL-TK. After 48 hr, cells were lysed and subjected to gene reporter assay.

Electrophoretic Mobility Shift Assay (EMSA)

Electrophoretic mobility shift assays was performed as described previously (Yamaoka et al., 1998). Briefly, nuclear extracts were prepared from HeLa cells as described previous (Yamaoka et al., 1998), and incubated with the radiolabeled probe in binding buffer (10 mM Tris-HCl pH7.5, 1 mM MgC12, 0.5 mM EDTA, 0.5 mM DTT, 50 mM NaCl, 200 ng/ml poly(dI-dC), 4% glycerol) containing end-labeled double-stranded NF-kB gel shift oligonucleotides (Santa Cruz) at 25° C. for 20 min. Samples were resolved on a 5% polyacrylamide native gel in 0.5× TBE, followed by autoradiography.

GST Pull-Down Assay, Immunoprecipitation and Immunoblotting Analyses

Cells were arrested at the G1/S phase or the mitotic phase, as describe previously. Total cells, 293T or HeLa, were lysed with GST-pulldown buffer (50 mM Hepes pH7.4, 150 mM NaCl, 10% glycerol, 1% Triton-X100, 1.5 mM MgC12, 1 mM EGTA, 100 mM NaF, 1 mM Na3VO4, 1 mM DTT and 0.5 μg/ml Leupeptin, 1.0 μg/ml Pepstatin, 0.2 mM PMSF) and incubated were incubated with 20 μl of agarose beads containing GST-Pin1 or GST at 4° C. for 2 hr, as described previously (Ryo et al., 2001). The precipitated proteins were washed three times with wash buffer containing and subjected to SDS-PAGE. For immunoprecipitation, cells were harvested at 24 hr after transfection and lysed with NP-40 lysis buffer (10 mM Tris HCl pH7.5, 100 mM NaCl, 0.5% NP-40, 1 mM Na3VO4, 0.5 μg/ml Leupeptin, 1.0 μg/ml Pepstatin, 0.2 mM PMSF). Cell lysates were incubated for 1 hr with Protein A/G Sepahrose/mouse IgG complexes.

Supernatant fraction was recovered and immunoprecipitated with 2 μg of anti-IkBα (Santa Cruz, sc-371) or anti-p65 (Santa Cruz, sc-109) antibodies and 30 μl Protein A/G sepharose. After washing three times with lysis buffer, pellets were analyzed on SDS-PAGE gels and immunoblotting analysis.

Ubiquitination Assay

Radio-labelled p65 protein was translated in vitro using the TNT coupled transcription/translation kit (Promega) in the presence of 8 μCi [35 S]-Met. Recombinant p65 truncation mutants were subclone into pGEX-KG vector and purified with glutathione beads column as described previously (Shen et al., 1998). For in vitro ubiquitination, 5 mg of GST-p65 proteins were added to 20 μl of in vitro ubiquitination reaction mix (1XERS, 30 mg/ml Rabbit E1, 160 mg/ml UbcH5a, 0.2 mg/ml ubiquitin, 5 mM ubiquitin aldehyde, 3.3 mg/ml HeLa S-100 extracts, 0.2 mM Lactacystin), followed by the incubation at 37° C. for 3 hr. Poly-ubiquitinated GST-p65 was purified with glutathione beads and subjected to immunoblot analysis with anti-ubiquitin antibody. For the ubiquitination of TNT-p65 protein, TNT p65 proteins were incubated with in vitro ubiquitination reaction mix without HeLa cell S-100 extracts at 37° C. for 2 hr, followed by SDS-PAGE analysis and autoradiography. For in vitro ubiquitination using 293T cell lysates, 293T cells were transfected either with SOCS-1, SOCS-IDS, or a control vector.

After 36 hrs, cell lysates were prepared by washing the cells twice with ice-cold PBS and lysing them in 250 μl of lysis buffer (20 mM HEPES [pH 7.2], 10 mM KCl, 1.5 mM MgCl2, 1 mM DTT, 25 μM MG-132, and protease and phosphatase inhibitors). Lysates were sonicated for two cycles of 30 s followed by centrifugation for 30 min. For the ubiquitination reaction, GST-p65 was resuspended in 50 μl of reaction buffer (1XERS, 10 mg/ml Rabbit E1, 80 mg/ml UbcH5a, 0.1 mg/ml ubiquitin, 2.5 mM ubiquitin alydehyde, 0.1 mM Lactacystin, 25 μM MG-132) containing 50 μg of cell lysates and then the suspension was incubated for 2 hr at 37° C. followed by the purification with glutahione beads and immunoblotting with anti-ubiquitin antibody.

Protein Degradation Assay

293T cells were transfected with Xpress-tagged p65. A plasmid encoding Xpress-LacZ was used as a transfection control. Cycloheximide (100 μg/ml) was added to the media 24 h after transfection to block continuing protein synthesis. Cells were harvested at each time points, and total lysates were analyzed by immunoblotting with anti-Xpress antibody (Invitrogene). The blots were scanned and semi-quantified by using the software NIH image 1.6.2, as described (Ryo et al., 2001). The results from three independent experiments are plotted such that the protein level at 0 h time point is 100%.

### Example 1

### Pin1 Levels Correlate with NF-B Activation in Human Breast Cancer Tissues

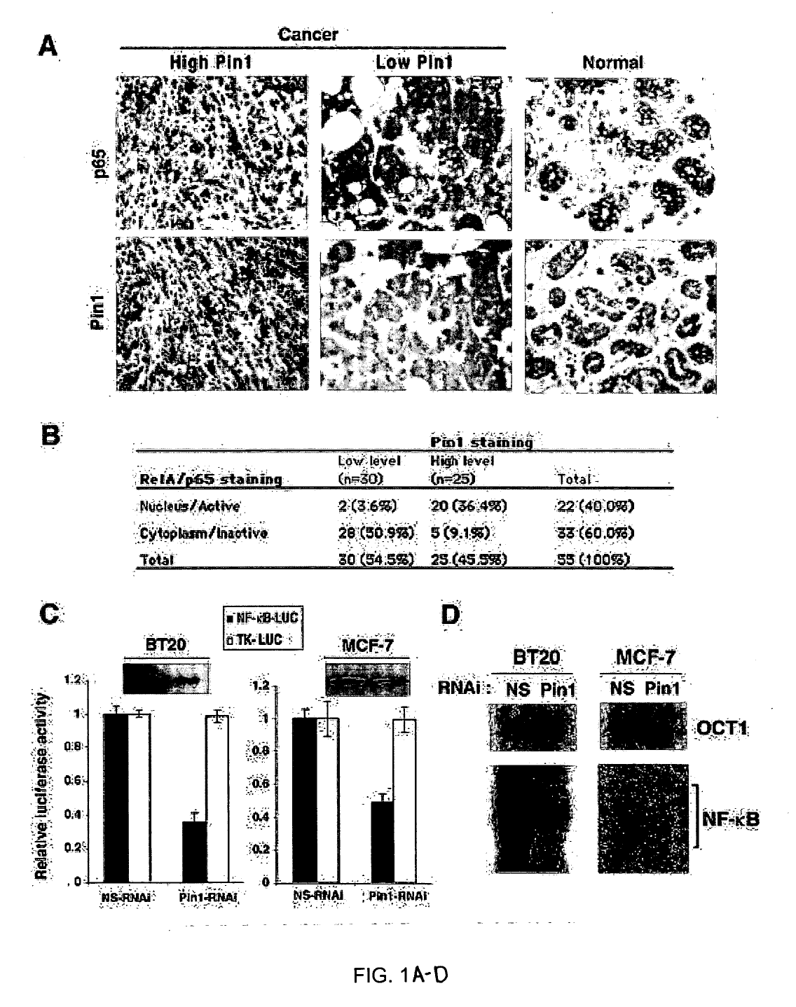

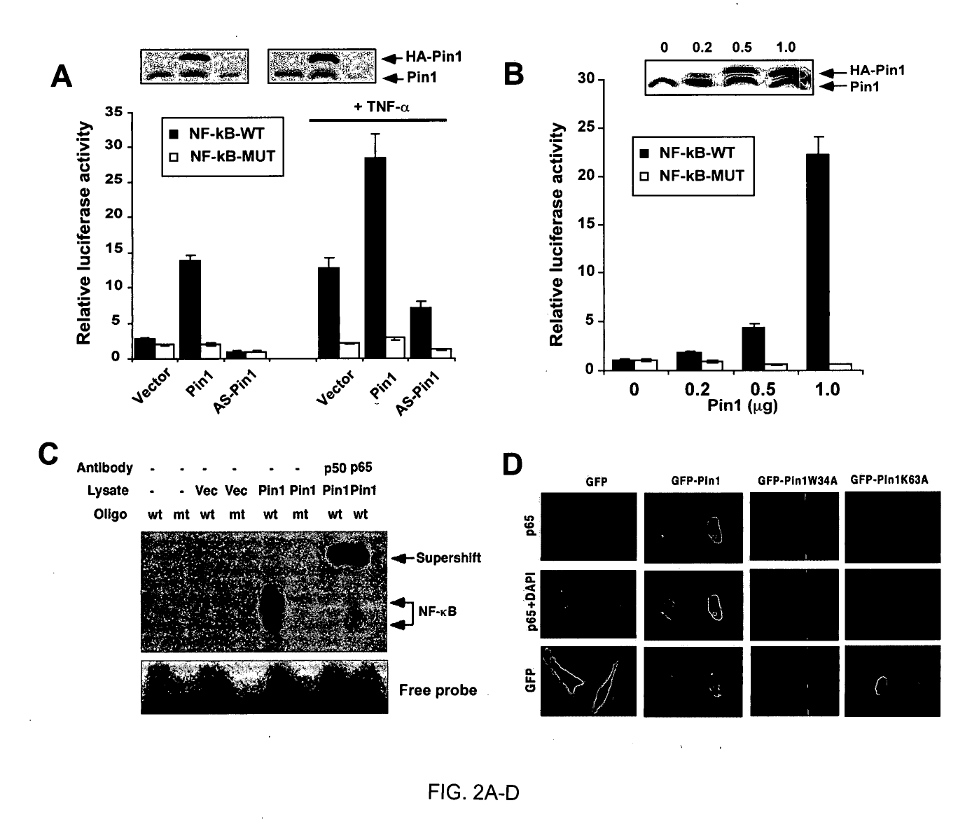

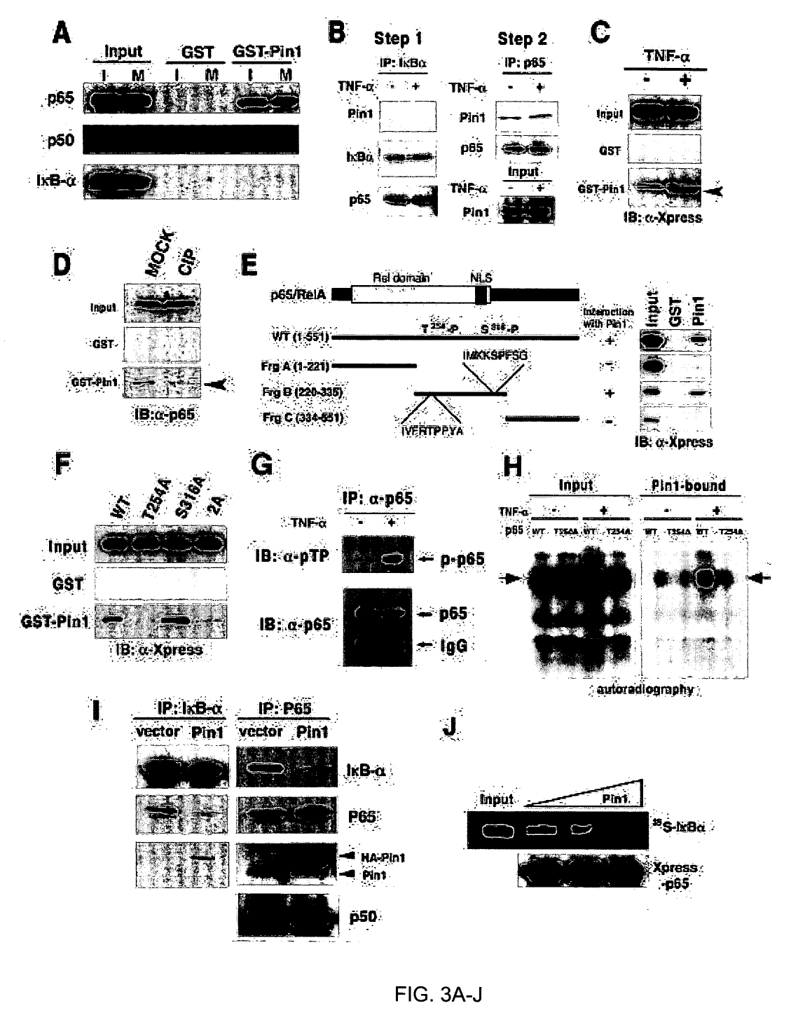

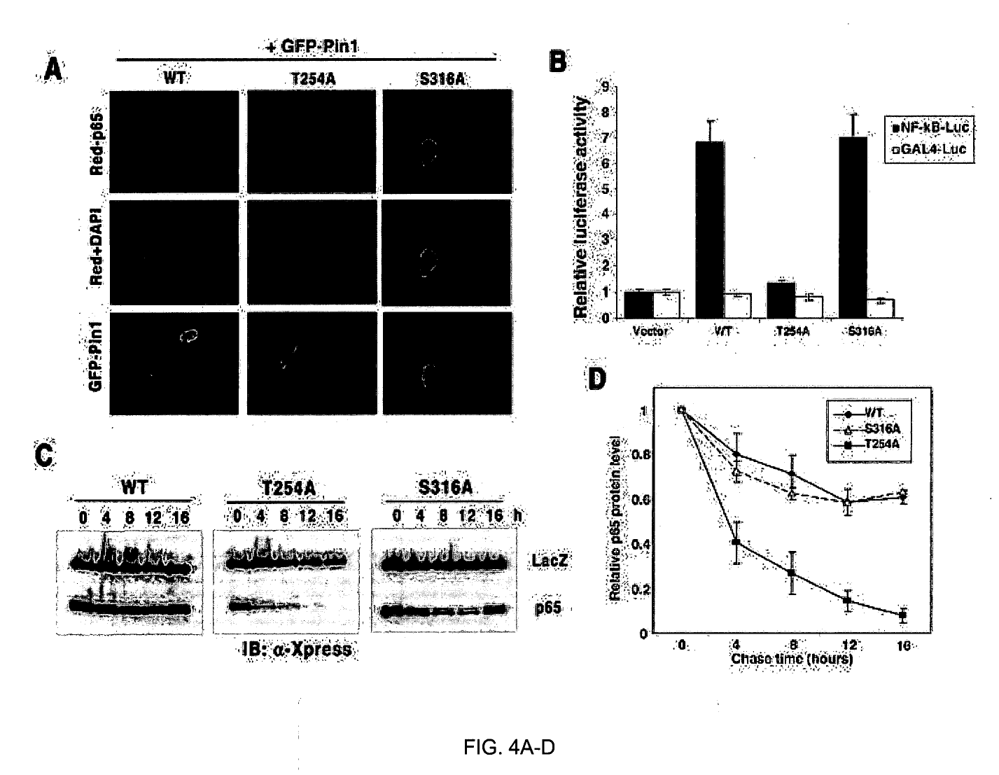

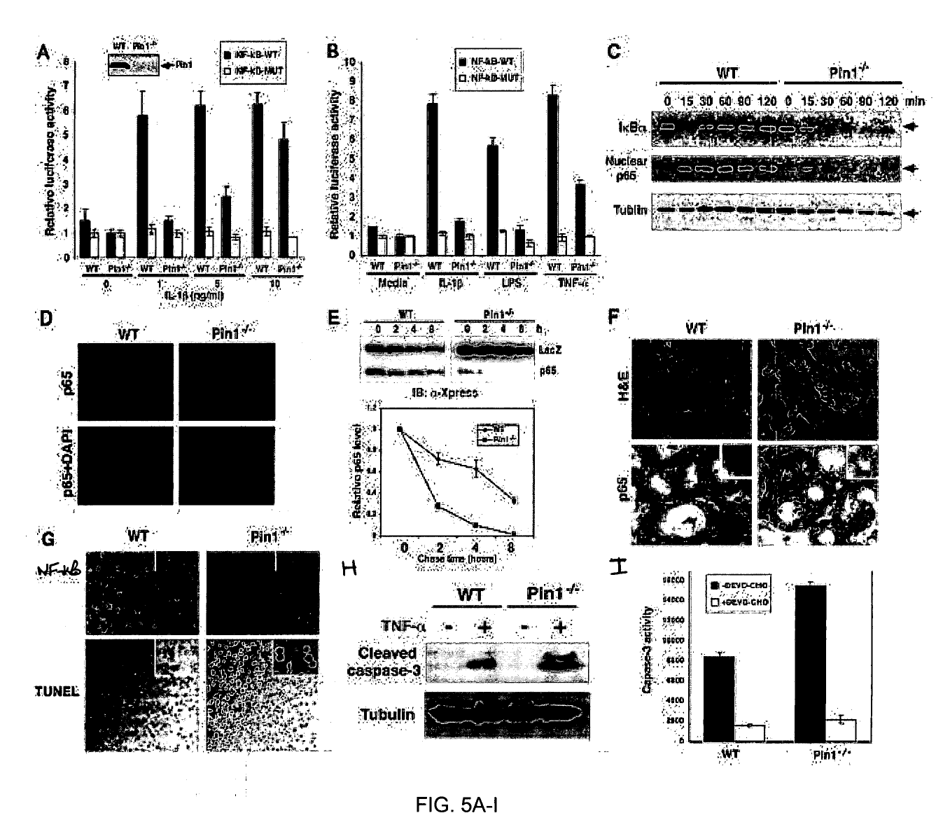

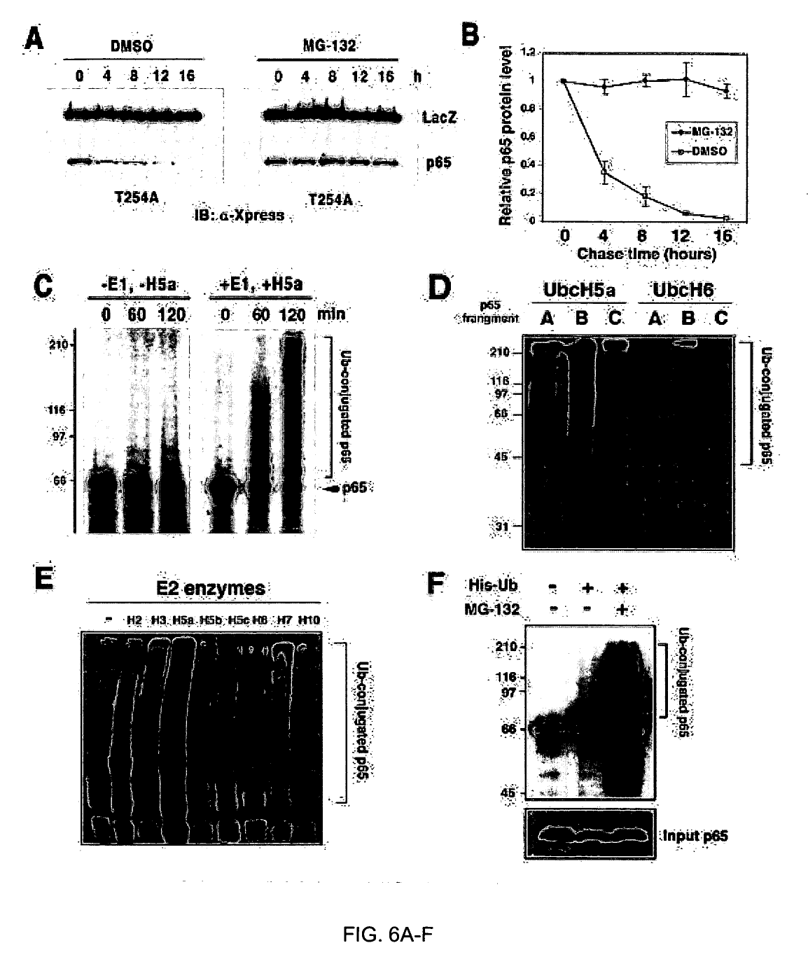

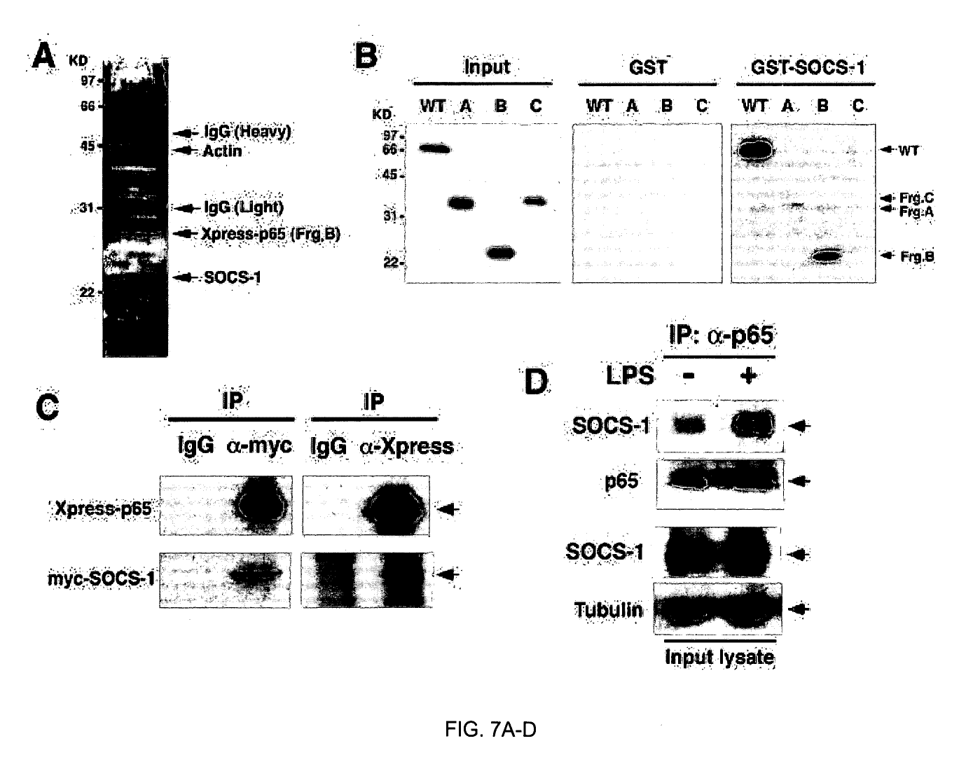

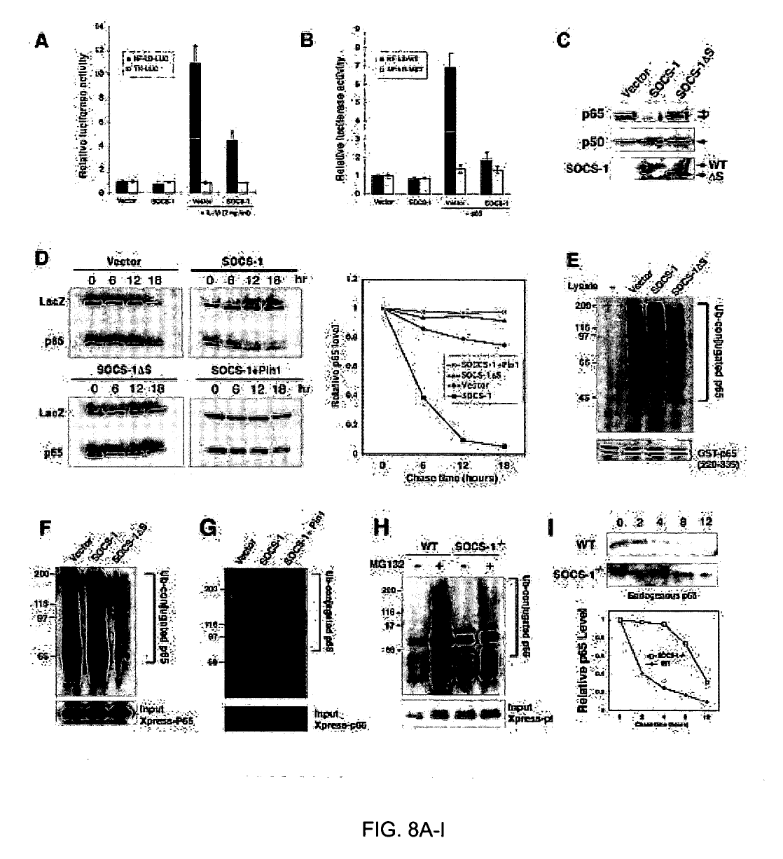

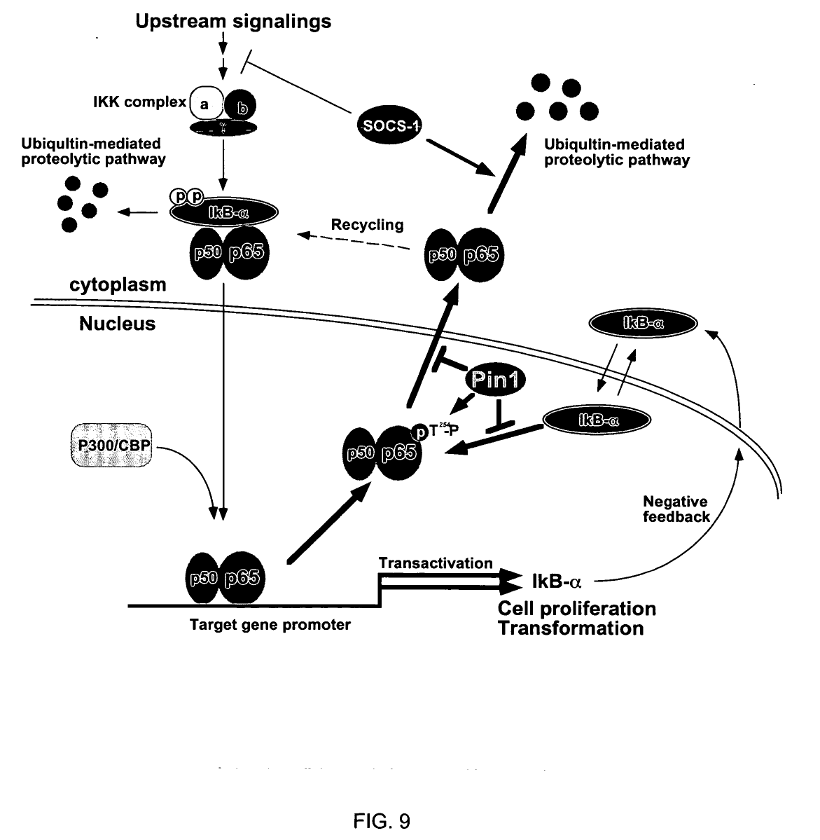

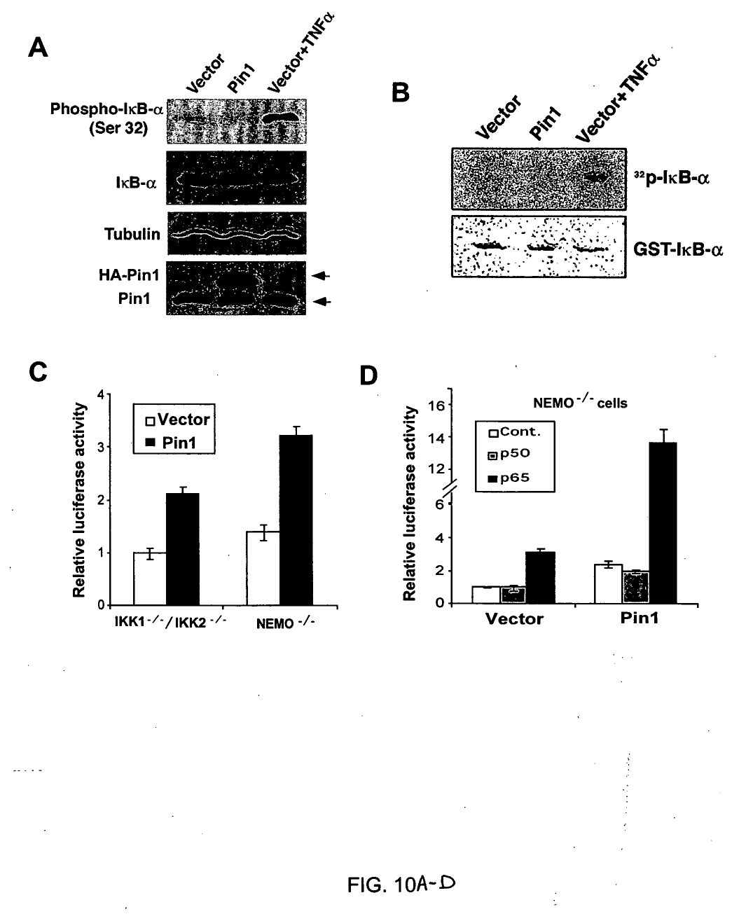

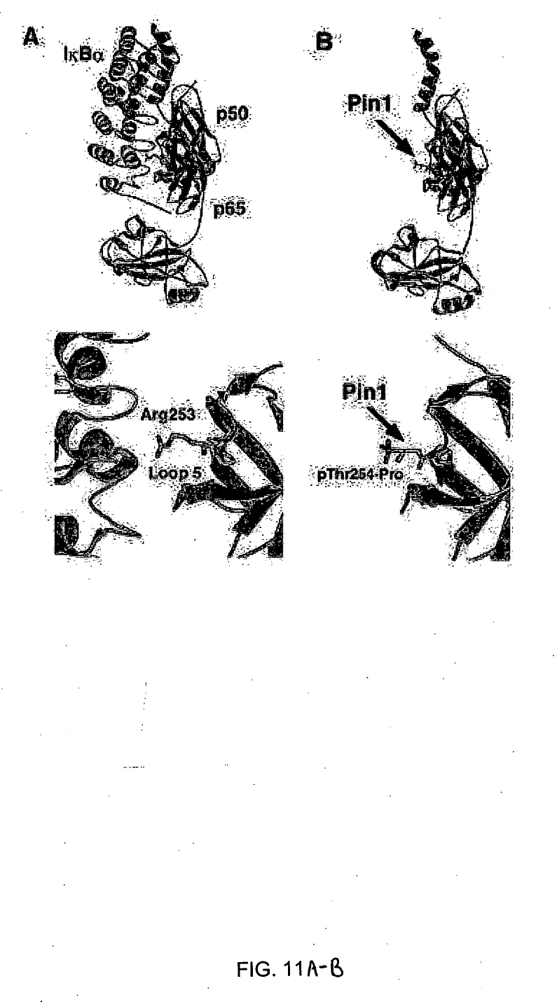

Both Pin1 and NF-κB have been shown to be highly activated in many human cancers (Baldwin, 2001; Karin et al., 2002; Ryo et al., 2002; Ryo et al., 2001; Wulf et al., 2001). Given that the NF-κB activation is regulated by a series of phosphorylation events, it was investigated whether Pin1 is involved in this regulation. To address this question, the correlation between Pin1 levels and NF-κB activation was examined in fifty human primary breast cancer samples and five normal breast tissues using immunohistochemitry. 20 out of 25 cancer samples containing high Pin1 levels had the strong nuclear accumulation of p65 protein, indicative of active NF-κB (FIGS. 1A, B). In contrast, 23 out of 25 cancer samples that contained low Pin1 levels exhibited cytoplasmic p65 localization, indicative of inactive NF-κB (FIGS. 1A, B). Each of 5 normal mammary gland samples contained low Pin1 levels and cytoplasmic p65 localization (FIGS. 1A, B). These results suggested a possible correlation between Pin1 levels and NF-κB activation. To further examine this possibility, the effects of Pin1 inhibition on NF-κB activity were determined in two breast cancer cell line BT20 (ER-negative) and MCF-7 (ER-positive), which have been shown to have constitutive activation of NF-κB (FIGS. 1C, D) (Nakshatri and Goulet, 2002). In both cell lines, inhibition of Pin1 by siRNA or antisense Pin1 construct (Pin1^{AS}) significantly suppressed the transcriptional activity of NF-κB-Luc, but not the control TK-Luc promoter reporter construct (FIG. 1C). Furthermore, gel shift assay confirmed that the RNAi treatment also suppressed the DNA binding activity of NF-kB, but not the control OCT-1 (FIG. 1D). These results indicate that Pin1 levels correlate with NF-κB activity in human breast cancer tissues and that inhibition of Pin1 suppresses NF-κB activation in cells.

### Example 2

### Pin1 Activates NF-B Signaling

The above results suggest that Pin1 activates NF-kB signaling. To further explore this possibility, the following three different assays were used. First, gene reporter assay to examine the effect of Pin1 on NF-kB transcriptional activity were performed. Overexpression of Pin1 activated, whereas depletion of endogenous Pin1 using Pin1 AS suppressed NF-kB transcriptional activity in a dose dependent manner (FIGS. 2A and B). Furthermore, Pin1 also cooperated with TNF-α to activate NF-kB activity (FIG. 2A). These results indicate that Pin1 enhances NF-kB transcriptional activity by cooperation with upstream signaling. Next, EMSA assays were performed to examine whether overexpression of Pin1 increases DNA binding activity of NF-kB in cells. The DNA binding activity of NF-kB was significantly increased in Pin1 transfected cells, but not in vector controls (FIG. 2C). Furthermore, these DNA-protein complexes were supershifted by either anti-p65 or -p50 antibodies (FIG. 2C), confirming that the NF-kB complexes consist of both p65 and p50. Finally, we performed an immunofluorescence study to examine the effects of Pin1 on subcellular localization of NF-kB. When DsRed-p65 was transfected with GFP control, it localized primarily in the cytoplasm. However, co-transfection with GFP-Pin1 prompted p65 to translocate into the nucleus. In contrast, neither its WW-domain nor PPIase mutants of Pin1 had this effect, indicating that both the pSer/Thr-Pro-binding and -isomerizing activities are required for Pin1 to modulate the subcellular localization of p65, as shown for all Pin1 substrates examined so far (Lu et al., 2002).

Together, the results of the above three assays indicate that Pin1 enhances NF-kB transcriptional activity by enhancing its nuclear translocation and DNA binding activity.

### Example 3

### Pin1 Dose not Alter the IKK Activity and Phosphorylation Status of IkB

Given that Pin1 enhances NF-kB transcriptional activity, a key question was to determine in which pathway Pin1 might exert its effect in NF-kB signaling. NF-kB has been shown to be activated by IKK mediated phosphorylation and subsequent degradation of the NF-kB inhibitor IkBa. This allows NF-kB to translocate into the nucleus to activate target genes (Baeuerle and Baltimore, 1996; Ghosh et al., 1998). To examine whether Pin1 affects NF-kB upstream regulators, we performed the IKK kinase assay and immunoblot analysis using a phospho-specific IkBa antibody (Ser32) to determine effects of Pin1 on the IKK activity and IkBα phosphorylation, respectively. Overexpression of Pin1 did not result in a detectable increase either in IKK kinase activity or IkBα phosphorylation, although both were increased by TNFα (FIGS. 10A, B). These results suggest that the effects of Pin1 on the activation of NF-kB might be independent of the IkBα phosphorylation by IKK. To further support these observations, the ability of Pin1 to activate NF-kB in IKK1−/−/IKK2−/− or NEMO−/− MEFs was examined, in which IKK activity is completely disrupted (Li et al., 2000; Rudolph et al., 2000).

Interestingly, Pin1 still enhanced NF-kB activity even in IKK1−/−IKK2−/− as well as in NEMO−/− cells (FIG. 1C). Notably, NF-kB was highly activated when NEMO−/− cells were co-transfected with Pin1 and p65, but not p50 (FIG. 10D), suggesting that p65 is the main Pin1 target. These results together support the connection that Pin1 affects the activation of NF-kB downstream of IKK phosphorylation of IkB.

### Example 4

### Pin1 Binds to p65 via the pThr254-Pro Motif and Inhibits its Interaction with IkB

Pin1 binds and isomerizes specific pSer/Thr-Pro motifs in certain phosphoproteins. To explore the molecular mechanism of NF-kB activation by Pin1, it was investigated as to whether any components of NF-kB or IkBα are Pin1 substrates. A well established procedure for this propose has been the GST-Pin1 pulldown assay (Lu et al., 1999; Shen et al., 1998; Yaffe et al., 1997). Although Pin1 did not bind to p50 or IkBα it specifically bound p65 from interphase and mitotic HeLa extracts (FIG. 3A). Furthermore, co-immunoprecipitation experiments also confirmed that endogenous Pin1 and p65 formed stable complexes in vivo (FIG. 3B). Moreover, this binding was almost completely abolished by the pre-treatment of the lysates with the phosphatase CIP (FIG. 3C), indicating that the binding is dependent on the phosphorylation of p65, as is the case for all known Pin1 substrates (Lu et al., 2002). Significantly, this binding was increased by ˜3 fold following the treatment with TNF-α(FIG. 3D), suggesting that this binding is enhanced by up-stream signaling for NF-κB activation. These results indicate that Pin1 specifically interacts with p65 in vitro and in vivo.

The Pin1 binding site(s) in p65, which contains only one Thr254-Pro motif and one Ser316-Pro motif and hence putative Pin1 binding sites was mapped (FIG. 3E). Three truncation mutants of p65 were generated and subjected them to the GST-pulldown assay. As expected, Pin1 specifically bound only to the truncated mutant B, which contains the two possible Pin1 binding sites (FIG. 3E). To determine which one Ser/Thr-Pro motif is required for Pin1 binding, a single Ala substitution into Thr254 or Ser316 in full length p65 protein was introduced. Pin1 bound the p65-S316A mutant, but completely failed to bind the p65-T254A mutant (FIG. 3F). To rule out the possibility that the lack of p65-T254A binding to Pin1 is due to its instability, transfected cells were treated with the proteasome inhibitor MG-132 before co-immunoprecipitation. Under these conditions, p65-T254A was stable and similar amounts of p65 proteins were immunoprecipitated, but Pin1 still failed to bind to p65-T254A mutant (FIG. 3G). These results indicate that Thr254 in p65 is necessary for Pin1 binding. To further support these results, a phospho-specific antibody that recognizes only the phosphorylated Thr-Pro motif was utilized. As shown in FIG. 3H, the pThr-Pro-specific antibody recognized only wild-type p65, but not its T254A mutant. Since p65 contains only single Thr-Pro motif, this result further support that Thr254-Pro in p65 is phosphorylated in vivo. These results are consistent with the previous findings that, as shown by in vivo ³²P labeling and phosphoamino acid analysis, p65 is phosphorylated on Thr residues in addition to Ser residues (Bird et al., 1997). Furthermore, the Thr phosphorylation of p65 is increased by a cytokine treatment, which is consistent with increased Pin1 binding to p65 following TNF-α treatment (FIG. 3D). Moreover, Thr254 is surrounded by Pin1 consensus binding sequences, consisting of multiple upstream hydrophobic residues (Ile, Val and Phe) and Pro residue an immediately downstream (Lu et al., 1999; Yaffe et al., 1997). These results collectively indicate that the Pin1-binding site in p65 is the Thr254-Pro motif.

Based on the crystal structure of the p65/p50 and IκBα complex, the Thr254-Pro motif in p65 is localized to near “hot spots”, which creates the binding interface for the interaction between p65 and IκBα (FIG. 11), suggesting that the binding and isomerization of the Thr254-Pro motif by Pin1 may interfere with the interaction of p65 with IκBα, but not with p50. To examine this possibility, cells were transfected with either Pin1 or control vectors and then subjected to immunoprecipitation with antibodies against p65 or IκBα. In cells overexpressing Pin1, significantly less p65 was detected in anti-IκBα immunoprecipitates (FIG. 3I). Similarly, less IκBα was immunoprecipitated by anti-p65 antibodies (FIG. 31). Furthermore, these differences were highly specific because overexpression of Pin1 had no detectable effect on the binding of p65 with p50 (FIG. 31). To further confirm this in vivo binding result, the effects of Pin1 on the binding between p65 and IκBα in vitro was examined. Cellular p65 was immunoprecipitated and incubated with ³⁵S-labeled IkBα in presence of increasing concentrations of Pin1. Pin1 inhibited the binding between p65 and IkBα in a dose-dependent manner (FIG. 3J). These results indicate that Pin1 binds to p65 phosphorylated on the Thr254-Pro motif and inhibits its interaction with IκBα in vitro and in vivo.

### Example 5

### The p65-T254A Mutant that act as a Pin1 Substrate is extremely Unstable and Fails to Transactivate NF-B Target Genes

To further investigate the biological significance of the interaction between Pin1 and p65, we first tested in vivo function of the p65-T254A mutant, which was unable to bind Pin1 (FIG. 3E). As a control, we used another mutant p65-S316A, which was able to bind Pin1 like WT p65 (FIG. 3E). When co-transfected with Pin1, both WT p65 and p65-S316A were localized in the nucleus, but the p65-T254A mutant could not be stabilized in the nucleus (FIG. 4A). Notably, the expression level of this mutant protein was found to be very low although mRNA expression was similar to that of WT p65 and its S316A mutant, suggesting the high protein turnover of the T254A mutant protein. Consistent with this result, the p65-T254A mutant failed to transactivate NF-kB downstream genes (FIG. 4A). These results suggest that Thr254 might be necessary for the nuclear localization and protein stability of p65. To directly assess this possibility, 293T cells were co-transfected with either Xpress-p65 or its site-directed mutants, together with Xpress-LacZ as an internal control. At 24 hr following transfection, cells were treated with cycloheximide to block protein synthesis, followed by a measurement of p65 protein stability via immunoblotting analysis, as described previously (Wulf et al., 2002). Although the half-life of the p65-S316A mutant was similar to that of the WT protein, the half-life of the p65-T254A mutant was considerably shorter (FIGS. 4B, C). The results indicate that the phosphorylation and subsequent Pin1 interaction at the Thr254-Pro motif is required for the protein stability and nuclear localization of p65.

### Example 6

### Pin1-Deficient Cells are Refractory to NF-κB Activation by Cytokine Signals due to Rapid p65 Nuclear Export and Degradation

The above results indicate that Pin1 overexpression inhibits p65 binding to IκBα and enhances its nuclear localization, protein stability and transactivation, and that the p65 mutant that cannot act as a Pin1 substrate is extremely unstable and fails to transactivate NF-κB target genes. Key questions are whether endogenous Pin1 is required for nuclear localization and protein stability of p65 as well as NF-κB signaling. To address these questions, primary mouse embryonic fibroblasts (MEFs) derived from Pin1 knockout (Pin1^{-/-}) and WT mice were used. In contrast to WT MEFs, Pin1^{-/-} cells were refractory to the activation of NF-κB when treated with moderate concentrations of IL-1β, but not with high doses (FIG. 5A). Furthermore, these cells were also resistant to NF-κB activation by TNF-α or LPS stimulation, which was not the case in WT cells (FIG. 5B). These results indicate that Pin1 is necessary for the activation of NF-κB in vitro.

To investigate the molecular mechanism underlying the resistance of Pin1^{-/-} MEFs to cytokine signaling, cells were treated with moderate concentration of IL-1β and examined the levels of IκBα and nuclear p65 at different time points. As shown previously (Bannerman et al., 2002), immediately following IL-1α treatment, IkBα was rapidly degraded and nuclear p65 levels were accumulated up to the 60 min time point in WT cells (FIG. 5C). This was followed by the up-regulation of IκBα after 60 min (FIG. 5C), which has previously been shown to be due to transactivation of IkBα gene by NF-κB (Beg et al., 1993; Brown et al., 1993; Chiao et al., 1994; Sun et al., 1993). However, in Pin1^{-/-} cells, IκBα was degraded immediately following IL-1β treatment, as in WT cells, but κBα levels were not up-regulated even after 120 min (FIG. 5C). Importantly, in these cells there was barely any nuclear accumulation of p65 following the degradation of IκBα (FIG. 5C). To further confirm these results, after IL-1α treatment p65 was immunostained for. Consistent with these immunoblotting data, p65 was readily detected in the nucleus of WT MEFs (FIG. 5D). However, in Pin1^{-/-} MEFs, p65 levels were not only much lower, but the protein was almost completely excluded from the nucleus (FIG. 5D).

To further determine the importance of Pin1 for maintaining p65 protein stability, both Xpress-tagged p65 and Xpress-LacZ into Pin1^{-/-} or WT MEFs were transfected and then monitored their protein stability, as described above. The stability of p65 protein was dramatically decreased in Pin1^{-/-} MEFs as compared with that in WT cells (FIG. 5E). Together with the above findings that the Pin1 binding site mutant p65-T254A is also extremely unstable in Pin1-positive 293Tcells (FIGS. 4C, D), these results indicate that the functional interaction between Pin1 and p65 are necessary for the nuclear localization and stability of p65.

Finally, to examine whether Pin1 is required for NF-κB activation in vivo, the effects of Pin1 knockout on NF-κB-related phenotypes in mice were determined. Although deletion of p65 in mice is lethal (Beg et al., 1995), disruptions of some NF-κB upstream regulators such as IKKα also affects mammary gland cell proliferation during pregnancy (Brantley et al., 2001; Cao et al., 2001). Since a similar mammary gland phenotype has been observed in Pin1^{-/-} mice (Liou et al., 2002), p65 protein levels and subcellular localization in WT and Pin1^{-/-} mouse mammary glands 1 day after delivery was compared. As shown (Brantley et al., 2001; Cao et al., 2001), NF-κB is activated, as indicated by the increase in its protein level and nuclear localization (FIG. 5F). However, in Pin1^{-/-} mice, NF-κB levels were very low and virtually excluded from the nucleus (FIG. 5F), indicating that NF-κB is inactive, suggesting that loss of Pin1 function might affect activation of NF-κB in vivo.

To further support this suggestion, the effects of Pin1 knockout on TNF-α-induced apoptosis in mice were examined. It has been shown that p65 knockout causes massive apoptosis in embryonic livers (Beg et al., 1995) and that inhibition of NF-kB sensitizes cells to TNF-α induced apoptosis in vitro and in vivo, including adult mouse liver cells (Chaisson et al., 2002; Van Antwerp et al., 1996). If Pin1 is important for activation of NF-kB in vivo, Pin1 knockout mice would be more susceptible to TNF-α induced liver apoptosis. To test this possibility, WT and Pin1^{-/-} mice were treated with TNF-α, and 3 hr later, examined NF-kB activation and apoptosis in livers, as shown previously (Chaisson et al., 2002). Following TNF-α treatment, NF-κB was induced and accumulated in the nucleus of WT livers, but not Pin1^{-/-} livers, as shown by immunocytochemistry (FIG. 5G). Furthermore, apoptosis was drastically increased in Pin1^{-/-} livers as compared that in WT controls, as shown by TUNEL assay that detects fragmented DNA in apoptotic cells (FIG. 5G). This increased apoptosis in Pin1^{-/-} livers was also confirmed by an increase in cacpase-3 activity as determined by both immunoblotting for cleaved and active cacpase-3 (FIG. 5H) and a fluorogenic cacpase-3 activity assay (FIG. 51). These results indicate that loss of Pin1 function in mice reduces the activation of NF-kB and increases apoptosis in livers in response to TNF-α. Interestingly, similar apoptotic phenotypes have been observed in livers of mutant IkBα transgenic mice where NF-κB is inhibited (Chaisson et al., 2002). Taken altogether, the above results indicate that Pin1 is necessary for the activation of NF-κB in responses to cytokine signals both in vitro and in vivo.

### Example 7

### p65 Protein Stability is Regulated by Ubiquitin-Mediated Proteolytic Pathway

The investigation into the role of Pin1 in NF-kB signaling led to the discovery of a critical role for p65 protein stability in regulating NF-kB function. Since p65 has not been previously shown to be unstable, it was critical to demonstrate that its stability is regulated by specific proteolytic pathway(s). Given that ubiquitin-mediated proteolytic pathway are major regulatory mechanisms that control many signaling molecules in the cell, it was investigated whether such a pathway is responsible for the degradation of p65. One well established procedure to answer this question is to compare protein stability in the absence or presence of the proteasome inhibitor MG-132 (Ku and Omary, 2000). MG-132 strikingly prolonged the half-life of p65-T254A protein (FIGS. 6A, B), suggesting the possible involvement of the ubiquitin-mediated proteolytic pathway in the regulation of p65 stability. To examine whether p65 is actually ubiquitinated in vitro, 35 S-labeled p65 protein was synthesized and incubated it with ubiquitin in the absence or presence of ubiquitin activating enzyme (E1) and ubiquitin conjugating enzyme (E2) UbcH5a. Clearly, 35 S-labeled p65 was poly-ubiquitinated in the presence of E1 and UbcH5a in a time-dependent manner (FIG. 6C).

To examine which region of the p65 molecule can be ubiquitinated, different GST-p65 fragments (FIG. 3D) were incubated with S100 HeLa cells extracts, ubiquitin, E1 and UbcH5a or UbcH6, and then purified GST-p65 fragments using GST beads, followed by immunoblot with anti-ubiquitin antibodies. Although neither the fragment A nor C was ubiquitinated, fragment B, which contains the Pin1 binding Thr254-pro motif (FIG. 3E), was poly-ubiquitinated with UbcH5a, but not with UbcH6 (FIG. 6D). To examine whether other E2 enzymes could catalyze the ubiquitination of p65 in vitro, we incubated recombinant GST-p65 fragment B with in vitro ubiquitination assay using different E2 enzymes. Out of the 8 E2 enzymes examined, only UbcH5a effectively conjugated multiple ubiquitin molecules into p65 (FIG. 6E). Finally to investigate the poly-ubiquitination of p65 in vivo, we co-transfected p65 and His-tagged ubiquitin into HeLa cells, followed by MG-132 treatment to inhibit the proteasome function. Cell lysates were collected and subjected to the pulldown analysis with Ni-agarose beads to isolate His-tagged ubiquitin-conjugated protein using a urea-containing buffer to remove any p65-associating proteins, followed by immunoblot analysis with anti-p65 antibody as described previously (Ku and Omary, 2000). In the presence of MG-132 and His-ubiquitin, p65 was substantially poly-ubiquitinated (FIG. 6F). However, no ubiquitinated p65 was detected in the absence of His ubiquitin and/or MG-132 (FIG. 6F), indicating the specificity of the in vivo ubiquitination assay. These results indicate that p65 protein stability is controlled by ubiquitin-mediated proteolysis.

### Example 8

### The Cytokine Signal Inhibitor SOCS-1 Associates with p65 and Regulates its Ubiquitin-Mediated Proteolysis

Given that p65 protein turnover is regulated by ubiquitin-mediated proteolysis, it was next attempted to identify the specific ubiquitin ligase involved. For this propose, we subjected MEFs expressing Xpress-His-double tagged p65 fragment B to Ni-agarose affinity chromatography, followed by immunoprecipitation with anti-Xpress antibody.

Immunoprecipitates were separated by PAGE gel and individual protein bands were collected. A protein with the molecular weight of ˜23 kDa that was preferentially pulled down by p65 was identified to be Suppressor of Cytokine Signaling 1 (SOCS-1) (FIG. 7A) (Endo et al., 1997; Naka et al., 1997; Ohya et al., 1997). To confirm the interaction between p65 and SOCS-1, a series of in vitro and in vivo binding assays was performed. First, a GST pulldown assay using different p65 fragments revealed that SOCS-1 binds fill length p65 as well as p65 truncation fragment B, but not fragment A or C (FIG. 7B). Interestingly, it is this same fragment that is poly-ubiquitinated and contains the Pin1 binding site in p65. Second, the binding between exogenously expressed Xpress-p65 and Myc-SOCS-1 was detected by co-immunoprecipitation experiments using either anti-Xpress or anti-Myc antibody (FIG. 7C). Finally, the endogenous association between p65 and SOCS-1 was also confirmed in primary mouse spleenocytes by coimmunoprecipitation using anti-p65 antibody (FIG. 7D). These results indicate that SOCS-1 binds to p65 both in vitro and in vivo, and its binding site in p65 is close to those for both ubiquitination and Pin1-binding. SOCS-1, a member of the suppressors of cytokine signaling (SOCS) family of proteins, has been shown to be the ubiquitin ligase for Jak2 and Vav (Frantsve et al., 2001; Kamizono et al., 2001), and the above results suggest that it might be a putative ubiquitin ligase for p65.

Consistent with this idea, the in vivo association between p65 and SOCS-1 was significantly enhanced at 4 hr following LPS treatment (FIG. 7D), correlating with the downregulation of NF-kB following LPS stimulation (data not shown). Furthermore, overexpression of SOCS-1 significantly inhibited NF-κB activation by IL-1β (FIG. 8A). Moreover, overexpression of SOCS-1 also significantly suppressed NF-κB activation induced by exogenous expression of p65 (FIG. 8B). These results indicate that overexpression of SOCS-1 suppresses NF-κB activity probably by downregulating p65. To determine whether SOCS-1 functions as a ubiquitin ligase for p65, the effects of SOCS-1 on p65 protein stability was examined. Transfection of SOCS-1 significantly reduced protein levels of endogenous p65, but not p50 (FIG. 8C). In contrast, an SOCS-1 mutant with the SOCS domain deleted (SOCS-1ΔS), which has been shown to function as a dominant-negative mutant in ubiquitination (Frantsve et al., 2001; Kamizono et al., 2001; Ungureanu et al., 2002), slightly increased protein levels of p65, but not p50 (FIG. 8C).

To confirm the effects of SOCS-1 and its mutant on p65 protein stability, either control vector, SOCS-1, SOCS-1ΔS or SOCS-1 and Pin1 were co-transfected into 293 cells, followed by monitoring of p65 protein stability after cycloheximide treatment. SOCS-1 significantly reduced the half-life of p65, whereas SOCS-1ΔS slightly enhanced the protein stability of p65, as compared to control cells (FIG. 8D). Furthermore, co-expression of Pin1 completely blocked SOCS-1 induced degradation of p65 (FIG. 8D), which is consistent with the above findings that Pin1 increased p65 stability. Together, these results indicate that SOCS-1 is critical in mediating protein degradation of p65 and this process can be blocked by Pin1.

Given the obvious effects of SOCS-1 and Pin1 on p65 stability, the affect the ubiquitination of p65 was investigated. To detect the SOCS-1-mediated ubiquitination of p65 in vitro, 293T cells were transfected with either SOCS-1, SOCS-1ΔS or control vector and then soluble cell lysates added to GST-p65 protein, followed by a GST pulldown to examine ubiquitination of GST-p65 using anti-ubiquitin antibody. p65 was poly-ubiquitinated by 293T cell extracts, which was significantly enhanced by overexpression of SOCS-1, but decreased by SOCS-1ΔS mutant (FIG. 8E). To detect the effects of SOCS-1 and Pin1 on ubiquitination of p65 in vivo, cells were co-transfected with Xpress-p65, His-tagged ubiquitin and SOCS-1, SOCS-1ΔS, or control vector in the presence or absence of Pin1, followed by MG-132 proteasome inhibition. Cell lysates were subjected to Ni-agarose pulldown and immunoblotting with anti-Xpress or anti-p65 antibodies. Similar to in vitro ubiquitination, p65 was ubiquitinated in vivo, which was significantly enhanced by overexpression of SOCS-1, but decreased by SOCS-1ΔS mutant (FIG. 8F). Furthermore, co-expression of Pin1 significantly blocked SOCS-1-induced ubiquitination of p65 (FIG. 8G), similar to its effects on p65 protein stability (FIG. 8D).

Finally to address whether endogenous SOCS-1 is important for regulating p65 stability, ubiquitination and protein stability of p65 in SOCS-1^{-/-} and WT MEFs was compared. Although p65 was less stable in WT MEFs than in 293 cells, p65 was not only less ubiquitinated, but also much more stable in SOCS-1^{-/-} MEFs, as compared with that in WT controls (FIGS. 8H, I), demonstrating a specific requirement of endogenous SOCS-1 for mediating ubiquitination and degradation of p65. Taken together, these results indicate that SOCS-1 not only binds p65, but also modulates the ubiquitination and degradation of p65 and this process is regulated by Pin1.

### REFERENCES

Baeuerle, P. A., and Baltimore, D. (1996). NF-kappa B: ten years after. Cell 87, 13-20.
Baldwin, A. S. (2001). Control of oncogenesis and cancer therapy resistance by the transcription factor NF-kappaB. J Clin Invest 107, 241-246.
Bannerman, D. D., Tupper, J. C., Kelly, J. D., Winn, R. K., and Harlan, J. M. (2002). The Fas-associated death domain protein suppresses activation of NF-kappa B by LPS and IL-1 beta. J Clin Invest 109, 419-425.
Beg, A. A., Finco, T. S., Nantermet, P. V., and Baldwin, A. S., Jr. (1993). Tumor necrosis factor and interleukin-1 lead to phosphorylation and loss of I kappa B alpha: a mechanism for NF-kappa B activation. Mol Cell Biol 13, 3301-3310.
Beg, A. A., Sha, W. C., Bronson, R. T., Ghosh, S., and Baltimore, D. (1995). Embryonic lethality and liver degeneration in mice lacking the RelA component of NF-kappa B. Nature 376, 167-170.
Bhat-Nakshatri, P., Sweeney, C. J., and Nakshatri, H. (2002). Identification of signal transduction pathways involved in constitutive NF-kappaB activation in breast cancer cells. Oncogene 21, 2066-2078.
Bird, T. A., Schooley, K., Dower, S. K., Hagen, H., and Virca, G. D. (1997). Activation of nuclear transcription factor NF-kappaB by interleukin-1 is accompanied by casein kinase II-mediated phosphorylation of the p65 subunit. J Biol Chem 272, 32606-32612.
Brantley, D. M., Chen, C. L., Muraoka, R. S., Bushdid, P. B., Bradberry, J. L., Kittrell, F., Medina, D., Matrisian, L. M., Kerr, L. D., and Yull, F. E. (2001). Nuclear factor-kappaB (NF-kappaB) regulates proliferation and branching in mouse mammary epithelium. Mol Biol Cell 12, 1445-1455.
Brown, K., Park, S., Kanno, T., Franzoso, G., and Siebenlist, U. (1993). Mutual regulation of the transcriptional activator NF-kappa B and its inhibitor, I kappa B-alpha. Proc Natl Acad Sci USA 90, 2532-2536. Cao, Y., Bonizzi, G., Seagroves, T. N., Greten, F. R., Johnson, R., Schmidt, E. V., and Karin, M. (2001). IKKalpha provides an essential link between RANK signaling and cyclin D1 expression during mammary gland development. Cell 107, 763-775.
Chaisson, M. L., Brooling, J. T., Ladiges, W., Tsai, S., and Fausto, N. (2002). Hepatocyte-specific inhibition of NF-kappaB leads to apoptosis after TNF treatment, but not after partial hepatectomy. J Clin Invest 110, 193-202.
Chen, L., Fischle, W., Verdin, E., and Greene, W. C. (2001). Duration of nuclear NF-kappaB action regulated by reversible acetylation. Science 293, 1653-1657.
Chiao, P. J., Miyamoto, S., and Verma, I. M. (1994). Autoregulation of I kappa B alpha activity. Proc Natl Acad Sci USA 91, 28-32.
De Sepulveda, P., Ilangumaran, S., and Rottapel, R. (2000). Suppressor of cytokine signaling-1 inhibits VAV function through protein degradation. J Biol Chem 275, 14005-14008.
DiDonato, J. A., Hayakawa, M., Rothwarf, D. M., Zandi, E., and Karin, M. (1997). A cytokine-responsive IkappaB kinase that activates the transcription factor NF-kappaB. Nature 388, 548-554.
Endo, T. A., Masuhara, M., Yokouchi, M., Suzuki, R., Sakamoto, H., Mitsui, K., Matsumoto, A., Tanimura, S., Ohtsubo, M., Misawa, H., Miyazaki, T., Leonor, N., Taniguchi, T., Fujita, T., Kanakura, Y., Komiya, S., and Yoshimura, A. (1997). A new protein containing an SH2 domain that inhibits JAK kinases. Nature 387, 921-924.
Frantsve, J., Schwaller, J., Sternberg, D. W., Kutok, J., and Gilliland, D. G. (2001). Socs-1 inhibits TEL-JAK2-mediated transformation of hematopoietic cells through inhibition of JAK2 kinase activity and induction of proteasome-mediated degradation. Mol Cell Biol 21, 3547-3557.
Ghosh, S., and Karin, M. (2002). Missing pieces in the NF-kappaB puzzle. Cell 109 Suppl, S81-96.
Ghosh, S., May, M. J., and Kopp, E. B. (1998). NF-kappa B and Rel proteins: evolutionarily conserved mediators of immune responses. Annu Rev Immunol 16, 225-260.
Huxford, T., Huang, D. B., Malek, S., and Ghosh, G. (1998). The crystal structure of the IkappaBalpha/NF-kappaB complex reveals mechanisms of NF-kappaB inactivation. Cell 95, 759-770.
Israel, A. (2000). The IKK complex: an integrator of all signals that activate NF-kappaB? Trends Cell Biol 10, 129-133.
Jacobs, M. D., and Harrison, S. C. (1998). Structure of an IkappaBalpha/NF-kappaB complex. Cell 95, 749-758.
Kamizono, S., Hanada, T., Yasukawa, H., Minoguchi, S., Kato, R., Minoguchi, M., Hattori, K., Hatakeyama, S., Yada, M., Morita, S., Kitamura, T., Kato, H., Nakayama, K., and Yoshimura, A. (2001). The SOCS box of SOCS-1 accelerates ubiquitin-dependent proteolysis of TEL-JAK2. J Biol Chem 276, 12530-12538.
Karin, M. (1999). How NF-kappaB is activated: the role of the IkappaB kinase (IKK) complex. Oncogene 18, 6867-6874.
Karin, M., Cao, Y., Greten, F. R., and Li, Z. W. (2002). NF-kappaB in cancer: from innocent bystander to major culprit. Nat Rev Cancer 2, 301-310.
Kile, B. T., Schulman, B. A., Alexander, W. S., Nicola, N. A., Martin, H. M., and Hilton, D. J. (2002). The SOCS box: a tale of destruction and degradation. Trends Biochem Sci 27, 235-241.
Kinjyo, I., Hanada, T., Inagaki-Ohara, K., Mori, H., Aki, D., Ohishi, M., Yoshida, H., Kubo, M., and Yoshimura, A. (2002). SOCS1/JAB is a negative regulator of LPS-induced macrophage activation. Immunity 17, 583-591.
Ku, N. O., and Omary, M. B. (2000). Keratins turn over by ubiquitination in a phosphorylation-modulated fashion. J Cell Biol 149, 547-552.
Li, Q., Estepa, G., Memet, S., Israel, A., and Verma, I. M. (2000). Complete lack of NF-kappaB activity in IKK1 and IKK2 double-deficient mice: additional defect in neurulation. Genes Dev 14, 1729-1733.
Li, Q., and Verma, I. M. (2002). NF-kappaB regulation in the immune system. Nat Rev Immunol 2, 725-734.
Lindeman, G. J., Wittlin, S., Lada, H., Naylor, M. J., Santamaria, M., Zhang, J. G., Starr, R., Hilton, D. J., Alexander, W. S., Ormandy, C. J., and Visvader, J. (2001). SOCS1 deficiency results in accelerated mammary gland development and rescues lactation in prolactin receptor-deficient mice. Genes Dev 15, 1631-1636.
Liou, Y. C., Ryo, R., Huang, H. K., Lu, P. J., Bronson, R., Fujimori, F., Uchidafl, U., Hunter, T., and Lu, K. P. (2002). Loss of Pin1 function in the mouse resembles the cyclin D1-null phenotypes. Proc. Natl. Acad. Sci. USA 99, 1335-1340.
Lu, K. P., Hanes, S. D., and Hunter, T. (1996). A human peptidyl-prolyl isomerase essential for regulation of mitosis. Nature 380, 544-547.
Lu, K. P., Liou, Y. C., and Zhou, X. Z. (2002). Pinning down proline-directed phosphorylation signaling. Trends Cell Biol 12, 164-172.
Lu, P. J., Zhou, X. Z., Shen, M., and Lu, K. P. (1999). A function of WW domains as phosphoserine- or phosphothreonine-binding modules. Science 283, 1325-1328.
Mercurio, F., Zhu, H., Murray, B. W., Shevchenko, A., Bennett, B. L., Li, J., Young, D. B., Barbosa, M., Mann, M., Manning, A., and Rao, A. (1997). IKK-1 and IKK-2: cytokine-activated IkappaB kinases essential for NF-kappaB activation. Science 278, 860-866.
Miyamoto, S., Chiao, P. J., and Verma, I. M. (1994). Enhanced I kappa B alpha degradation is responsible for constitutive NF-kappa B activity in mature murine B-cell lines. Mol Cell Biol 14, 3276-3282.
Naka, T., Narazaki, M., Hirata, M., Matsumoto, T., Minamoto, S., Aono, A., Nishimoto, N., Kajita, T., Taga, T., Yoshizaki, K., Akira, S., and Kishimoto, T. (1997). Structure and function of a new STAT-induced STAT inhibitor. Nature 387, 924-929.
Nakagawa, R., Naka, T., Tsutsui, H., Fujimoto, M., Kimura, A., Abe, T., Seki, E., Sato, S., Takeuchi, O., Takeda, K., Akira, S., Yamanishi, K., Kawase, I., Nakanishi, K., and Kishimoto, T. (2002). SOCS-1 participates in negative regulation of LPS responses. Immunity 17, 677-687.
Nakshatri, H., and Goulet, R. J., Jr. (2002). NF-kappaB and breast cancer. Curr Probl Cancer 26, 282-309.
Perkins, N. D., Felzien, L. K., Betts, J. C., Leung, K., Beach, D. H., and Nabel, G. J. (1997). Regulation of NF-kappaB by cyclin-dependent kinases associated with the p300 coactivator. Science 275, 523-527.
Ranganathan, R., Lu, K. P., Hunter, T., and Noel, J. P. (1997). Structural and functional analysis of the mitotic peptidyl-prolyl isomerase Pin1 suggests that substrate recognition is phosphorylation dependent. Cell 89, 875-886.
Regnier, C. H., Song, H. Y., Gao, X., Goeddel, D. V., Cao, Z., and Rothe, M. (1997). Identification and characterization of an IkappaB kinase. Cell 90, 373-383.
Rottapel, R., Ilangumaran, S., Neale, C., La Rose, J., Ho, J. M., Nguyen, M. H., Barber, D., Dubreuil, P., and de Sepulveda, P. (2002). The tumor suppressor activity of SOCS-1. Oncogene 21, 4351-4362.
Rudolph, D., Yeh, W. C., Wakeham, A., Rudolph, B., Nallainathan, D., Potter, J., Elia, A. J., and Mak, T. W. (2000). Severe liver degeneration and lack of NF-kappaB activation in NEMO/IKK-deficient mice. Genes Dev 14, 854-862.
Ryo, A., Liou, Y. C., Lu, K. P., and Wulf, G. (2003). Prolyl isomerase Pin1: a catalyst for oncogenesis and a potential therapeutic target in cancer. J Cell Sci 116, 773-783.
Ryo, A., Liou, Y. C., Wulf, G., Nakamura, M., Lee, S. W., and Lu, K. P. (2002). PIN1 Is an E2F Target Gene Essential for Neu/Ras-Induced Transformation of Mammary Epithelial Cells. Mol Cell Biol 22, 5281-5295.
Ryo, A., Nakamura, N., Wulf, G., Liou, Y. C., and Lu, K. P. (2001). Pin1 regulates turnover and subcellular localization of beta-catenin by inhibiting its interaction with APC. Nature Cell Biol 3, 793-801.
Sen, R., and Baltimore, D. (1986). Inducibility of kappa immunoglobulin enhancer-binding protein Nf-kappa B by a posttranslational mechanism. Cell 47, 921-928.
Shen, M., Stukenberg, P. T., Kirschner, M. W., and Lu, K. P. (1998). The essential mitotic peptidyl-prolyl isomerase Pin1 binds and regulates mitosis-specific phosphoproteins. Genes Dev. 12, 706-720.
Sun, S. C., Ganchi, P. A., Ballard, D. W., and Greene, W. C. (1993). NF-kappa B controls expression of inhibitor I kappa B alpha: evidence for an inducible autoregulatory pathway. Science 259, 1912-1915.
Ungureanu, D., Saharinen, P., Junttila, I., Hilton, D. J., and Silvennoinen, O. (2002). Regulation of Jak2 through the ubiquitin-proteasome pathway involves phosphorylation of Jak2 on Y1007 and interaction with SOCS-1. Mol Cell Biol 22, 3316-3326.
Van Antwerp, D. J., Martin, S. J., Kafri, T., Green, D. R., and Verma, I. M. (1996). Suppression of TNF-alpha-induced apoptosis by NF-kappaB. Science 274, 787-789.
Wulf, G. M., Liou, Y. C., Ryo, A., Lee, S. W., and Lu, K. P. (2002). Role of Pin1 in the regulation of p53 stability and p21 transactivation, and cell cycle checkpoints in response to DNA damage. J Biol Chem 277, 47976-47979.
Wulf, G. M., Ryo, A., Wulf, G. G., Lee, S. W., Niu, T., and Lu, K. P. (2001). Pin1 is overexpressed in breast cancer and potentiates the transcriptional activity of phosphorylated c-Jun towards the cyclin D1 gene. EMBO J. 20, 3459-3472.
Yaffe, M. B., Schutkowski, M., Shen, M., Zhou, X. Z., Stukenberg, P. T., Rahfeld, J., Xu, J., Kuang, J., Kirschner, M. W., Fischer, G., Cantley, L. C., and Lu, K. P. (1997). Sequence-specific and phosphorylation-dependent proline isomerization: A potential mitotic regulatory mechanism. Science 278, 1957-1960.
Yamaoka, S., Courtois, G., Bessia, C., Whiteside, S. T., Weil, R., Agou, F., Kirk, H. E., Kay, R. J., and Israel, A. (1998). Complementation cloning of NEMO, a component of the IkappaB kinase complex essential for NF-kappaB activation. Cell 93, 1231-1240.
Yoshikawa, H., Matsubara, K., Qian, G. S., Jackson, P., Groopman, J. D., Manning, J. E., Harris, C. C., and Herman, J. G. (2001). SOCS-1, a negative regulator of the JAK/STAT pathway, is silenced by methylation in human hepatocellular carcinoma and shows growth-suppression activity. Nat Genet 28, 29-35.
Zandi, E., Rothwarf, D. M., Delhase, M., Hayakawa, M., and Karin, M. (1997). The IkappaB kinase complex (IKK) contains two kinase subunits, IKKalpha and IKKbeta, necessary for IkappaB phosphorylation and NF-kappaB activation. Cell 91, 243-252.
Zheng, H., You, H., Zhou, X. Z., Murray, S. A., Uchida, T., Wulf, G., Gu, L., Tang, X., Lu, K. P., and Xiao, Z. X. (2002). The prolyl isomerase Pin1 is a regulator of p53 in genotoxic response. Nature 419, 849-853.
Zhong, H., SuYang, H., Erdjument-Bromage, H., Tempst, P., and Ghosh, S. (1997). The transcriptional activity of NF-kappaB is regulated by the IkappaB-associated PKAc subunit through a cyclic AMP-independent mechanism. Cell 89, 413-424.
Zhong, H., Voll, R. E., and Ghosh, S. (1998). Phosphorylation of NF-kappa B p65 by PKA stimulates transcriptional activity by promoting a novel bivalent interaction with the coactivator CBP/p300. Mol Cell 1, 661-671.

1. A method of modulating the activity of an NF-kB polypeptide in a cell comprising contacting the cell with a substance that modulates the activity of Pin1 such that the activity of NF-kB is regulated.

2. The method of claim 1, wherein the ability of NF-kB to interact with IkBα is modulated.

3. The method of claim 1, wherein the peptidyl prolyl isomerase activity of Pin1 is modulated

4. The method of claim 1, wherein the substance is selected from the group consisting of a peptide, a peptide mimetic, a small molecule, and an antibody.

5. The method of claim 4, wherein said antibody is a monoclonal antibody.

6. A method of inhibiting the isomerization of the pThr254-Pro bond of the P65 subunit of NF-kB in a cell comprising contacting the cell with a substance that inhibits the activity of Pin1.

7. The method of claim 7 wherein said Pin1 activity is inhibited by contacting said Pin1 polypeptide with a substance that binds to the Pin1 active site.

8. The method of claim 7 wherein said Pin1 activity is inhibited by contacting said Pin1 polypeptide with a substance that binds to the WW domain.

9. The method of claim 7 or 8, where said substance is a small molecule.

10. The method of claim 7 or 8, where said substance is a peptide.

11. The method of claim 10, wherein said substance is a phosphoserine peptide.

12. The method of claim 7 or 8, where said substance is a peptide mimetic.

13. A method of inhibiting the isomerization of the pThr254-Pro bond in the P65 subunit of NF-kB said method comprising inhibiting the interaction of Pin1 and NF-kB.

14. The method of claim 13, where said compound is a small molecule.

15. The method of claim 13, where said compound is a peptide.

16. The method of claim 13, where said compound is a peptide mimetic.

17. A method of treating a subject suffering with a NF-kB associated condition comprising administering to said subject a Pin1 modulator thereby treating said subject.

18. The method of claim 17, wherein said NF-kB disorder is selected from a group consisting of a cell proliferative disorder, an immune response disorder, and an inflammatory disorder.

19. The method of claims 18, wherein said disorder is a cell proliferative disorder.

20. The method of claim 19 wherein said cell proliferative disorder is cancer.

21. The method of claim 20, wherein said cancer is breast cancer.

22. A method of treating a subject suffering from a NF-kB associated condition comprising administering said subject an antibody specific for an epitope comprising amino acid residues 254 and 255 of the p65 subunit of NF-kB, thereby treating said subject.

23. The method of claim 21, wherein said antibody is a monoclonal antibody.

24. The method of claim 22, wherein said antibody is a humanized antibody.

25. A method of increasing the amount of NF-kB proteolysis in a cell comprising the step of inhibiting the production of Pin1 thereby allowing NF-kB to be proteolyzed by the ubiquitin mediated proteolysis pathway.

26. The method of claim 24, wherein said inhibition of Pin1 production is by siRNA.

27. The method of claim 24, wherein said inhibition of Pin1 production is by RNAi.

28. A method of treating a subject suffering from a NF-kB associated disorder comprising administering said subject a compound that stimulates the expression of SOCS-1, thereby inhibiting the degredation of NF-kB.
